(19) United States
(12) Patent Application Publication
(10) Pub. No.: US 2026/0021043 A1
(43) Pub. Date: Jan. 22, 2026

(54) STARCH-BASED HYDROGEL FOR ADMINISTRATION OF THERAPEUTIC ANTICANCER AGENTS

(71) Applicant: Elizabeth Diana Barker, Knoxville, TN (US)

(72) Inventor: Elizabeth Diana Barker, Knoxville, TN (US)

(21) Appl. No.: 19/271,610

(22) Filed: Jul. 16, 2025

Related U.S. Application Data
(60) Provisional application No. 63/672,066, filed on Jul. 16, 2024.

(51) Int. Cl. A61K 9/06; A61K 31/704; A61K 41/00; A61K 47/20; A61K 47/36; A61K 51/12

(52) U.S. Cl. CPC A61K 9/06; A61K 31/704; A61K 41/0095; A61K 47/20; A61K 47/36; A61K 51/1213; A61K 2121/00

(57) ABSTRACT

A starch-based injectable hydrogel system for localized cancer treatment. The pre-formed hydrogel delivers chemotherapeutic drugs (such as doxorubicin) and/or medical radionuclides directly to tumor sites while minimizing systemic exposure. The composition uses dimethyl sulfoxide as a solvent and can be stored in pre-filled syringes for clinical use. Key advantages include >90% drug retention, sustained local delivery, and compatibility with image-guided injection techniques. The system is particularly suitable for pediatric brain tumors and other solid tumors requiring localized high-dose therapy.

### FIELD OF INVENTION

The present invention generally relates to methods and compositions comprising hydrogel structures. The invention particularly relates to starch-based injectable hydrogels for local administration of therapeutic agents. The invention further relates to formulations comprising chemotherapeutic and/or radiotherapeutic agents for intratumoral delivery to solid tumors.

### BACKGROUND

Chemotherapy remains a mainstay of cancer treatment. Chemotherapy is limited by systemic toxicity, poor tumor penetration, and/or heterogeneous drug distribution, within solid tumors. Current systemic delivery methods, such as intravenous (IV) administration may result in widespread drug exposure and/or off-target side effects, especially in pediatric patients. Tumor vasculature is often irregular and/or insufficiently perfused, preventing effective drug delivery to all regions of tumor tissue. The limitations mentioned herein are exacerbated in pediatric cancers where developmental factors alter pharmacokinetics, and/or cumulative systemic toxicity can severely impair long-term health outcomes.

A major unmet need in pediatric and/or adult oncology is the development of safe, effective local drug delivery systems that can penetrate solid tumor tissue while minimizing systemic toxicity. Existing hydrogel systems are limited in their ability to retain lipophilic chemotherapeutic agents, sequester therapeutic radionuclides, and/or adapt to complex tumor geometries. The hydrogel systems fail to provide sufficient encapsulation efficiency, mechanical integrity, and/or compatibility with injectable formulations. In particular, current hydrogel technology may not effectively combine local delivery of both chemotherapy and/or radiotherapy, within a single biocompatible injectable matrix.

### SUMMARY

In some aspects, the present disclosure relates to compositions and methods for localized delivery of therapeutic agents using a starch-based hydrogel system. The disclosed hydrogel is synthesized using amylopectin polymers in a dimethyl sulfoxide (DMSO) solvent system and may comprise one or more therapeutic agents. In some aspects, the hydrogel comprises a chemotherapeutic agent such as doxorubicin. The hydrogel may comprise one or more radionuclides selected from the list of therapeutic radionuclides.

The hydrogel is pre-formed prior to administration and is stored in a pre-filled syringe suitable for image-guided intratumoral injection. The composition is provided in a single-use format for local treatment of solid tumors in pediatric and/or adult patients.

The invention addresses the need for safe, localized delivery of chemotherapeutic and/or radiotherapeutic agents without systemic exposure. The disclosed hydrogel composition achieves high encapsulation efficiency, prolonged intratumoral retention, and/or formulation compatibility with hydrophobic drugs and unchelated radionuclides. In some aspects, the hydrogel retains greater than 90% of the encapsulated agent under physiological conditions for at least 7 days. In some aspects, systemic radioactivity is below detectable levels at 24 hours post-injection. The composition is administered to a solid tumor, including pediatric brain tumors, and is applied during or after surgical resection. The disclosed system enables single-agent or combination delivery to improve tumor control while minimizing off-target effects.

In some aspects, the invention provides a composition comprising a starch-based hydrogel comprising a network of starch polymer chains crosslinked using a crosslinking agent, a solvent comprising dimethyl sulfoxide, and a chemotherapeutic agent. The chemotherapeutic agent comprises doxorubicin in its free base form. The composition is a pre-formed injectable hydrogel formulation that is formed prior to administration.

In an aspect, the composition retains at least about 90% of the chemotherapeutic agent upon formation. The encapsulation efficiency is determined by ultraviolet-visible spectroscopy or dialysis analysis. The starch polymer chains are crosslinked using d-glucaric acid.

In an aspect, the composition retains at least about 90% of the chemotherapeutic agent under physiological conditions for a period of at least about 7 days. The retention is determined by dialysis or static diffusion testing. The composition is maintained under aqueous conditions at 37° C. during the retention analysis.

In an aspect, the composition is sterilized and stored in a pre-filled syringe suitable for intratumoral injection. The syringe is configured for single use and radiation-shielded storage. The composition remains physically stable in the syringe for at least about four weeks. The syringe is compatible with image-guided delivery techniques.

In an aspect, systemic exposure to the chemotherapeutic agent is below detectable levels at 24 hours following administration of the composition. The systemic exposure is assessed by mass spectrometry, high-performance liquid chromatography, or plasma fluorescence detection. The composition is administered via intratumoral injection to a solid tumor.

In an aspect, the composition is configured for administration to a pediatric solid tumor. The pediatric solid tumor comprises a brain tumor. The brain tumor comprises a medulloblastoma or an ependymoma. The composition is administered locally during or after surgical resection of the tumor.

In an aspect, the starch polymer chains comprise a blend of amylopectin molecules of differing molecular weight. The starch polymer chains comprise a ratio of high molecular weight to low molecular weight amylopectin in the range of from about 1:1 to about 1:5. The polymer blend is selected to modulate hydrogel viscosity or tissue penetration characteristics.

In an aspect, the composition is a pre-formed injectable hydrogel and does not require in situ polymerization prior to use. The composition is loaded into a sealed syringe barrel under aseptic manufacturing conditions.

In some aspects, the invention provides a composition comprising a starch-based hydrogel comprising a network of starch polymer chains crosslinked using a crosslinking agent, a solvent comprising dimethyl sulfoxide, and a medical radionuclide.

The composition is a pre-formed injectable hydrogel formulation that is formed prior to administration.

In an aspect, the hydrogel is sterilized and stored in a pre-filled syringe suitable for intratumoral injection. The syringe is configured for single use and radiation-shielded storage. The composition remains physically stable in the syringe for at least about four weeks. The syringe is compatible with image-guided delivery techniques.

In an aspect, systemic radioactivity resulting from administration of the composition is below detectable levels at 24 hours post-injection. The systemic radioactivity is assessed by gamma counting, scintigraphy, or radiometric detection of blood plasma. The radionuclide comprises cerium-144. The composition is administered via intratumoral injection to a solid tumor.

In an aspect, the composition is configured for administration to a pediatric solid tumor. The pediatric solid tumor comprises a brain tumor. The brain tumor comprises a medulloblastoma or ependymoma. The composition is administered locally during or after surgical resection of the tumor. In an aspect, the radionuclide is a medical radionuclide (as defined below). The radionuclide is selected based on its emission of alpha particles, beta particles, gamma particles and species for neutron capture or Auger electrons. The radionuclide may be selected based on its emission of alpha particles, beta particles, gamma photons, Auger electrons, and/or neutron-capture activation products. In some embodiments, the composition comprises a neutron-capture species such as a boron-containing compound or a gadolinium radionuclide. The composition is configured for site-specific delivery to a solid tumor and may retain the radionuclide without covalent tethering or chelator complexation. In some embodiments, a chelating agent may optionally be used to stabilize radionuclides that exhibit weak affinity for the hydrogel matrix; however, the composition may preferably enable chelator-free encapsulation under physiological conditions.

In an aspect, the composition further comprises a chemotherapeutic agent. The chemotherapeutic agent comprises doxorubicin in its free base form. The hydrogel retains at least about 90% of each therapeutic agent upon formation. The composition delivers the agents simultaneously to a single tumor site.

In an aspect, the composition is configured to enable monitoring of therapeutic agent distribution using imaging methods. The radionuclide comprises a gamma-emitting or positron-emitting radionuclide. The distribution of the therapeutic agent is assessed by scintigraphy, PET, or SPECT imaging. The composition provides a localized imaging signal at the site of administration.

In an aspect, the composition comprises a starch-based hydrogel loaded with actinium-225. The composition is administered via intratumoral injection to a solid tumor in a pediatric patient.

In some aspects, a method of treating a solid tumor comprises administering a composition comprising a starch-based hydrogel loaded with a chemotherapeutic agent, a radionuclide, or a combination thereof. The composition is administered via intratumoral injection into a tumor region. The composition is administered in a pre-formed state using a pre-filled syringe. The method results in reduction of tumor size, enhancement of local therapeutic concentration, or suppression of systemic toxicity.

In another aspect, a system comprises a therapeutic composition, a delivery apparatus, and a protocol for intratumoral administration. The delivery apparatus comprises a pre-filled syringe containing a starch-based hydrogel formed prior to administration.

In another aspect, a device comprises a sealed injection system containing the hydrogel composition disclosed herein. The device is compatible with radiation-shielded storage and sterile clinical use. The device is configured for administration of chemotherapeutic or radiotherapeutic agents directly to a tumor region.

In another aspect, the composition disclosed herein is administered using image-guided injection methods. The administration method comprises injection through a defined trajectory to a solid tumor region. The method comprises pre-formed hydrogel delivery and post-injection tumor response assessment.

In another aspect, the disclosed composition is suitable for storage and distribution as a pre-filled syringe-based injectable formulation.

In another aspect, the invention comprises methods, systems, compositions, and injectable delivery formats for localized, sustained release of chemotherapeutic or radiotherapeutic agents to solid tumors while minimizing systemic exposure.

In another aspect, the invention comprises a therapeutic system comprising a drug-loaded hydrogel, a delivery mechanism for localized administration, and a solid tumor as the treatment target. The system is configured to deliver one or more therapeutic agents to a defined anatomical site without systemic distribution.

In another aspect, the invention comprises a composition-storage system comprising a pre-filled syringe containing a starch-based hydrogel formulation. The syringe is compatible with image-guided or open-field injection to tumor tissue. The syringe remains sealed under sterile conditions and enables single-use clinical administration.

In another aspect, the invention comprises a therapeutic composition comprising one or more medical radionuclides selected from actinium-225, lutetium-177, radium-223, bismuth-213, lead-212, terbium-149, fermium-225, thorium-227, thorium-226, astatine-211, astatine-217, uranium-230, iodine-131, iridium-192, iodine-125, palladium-103, cesium-131, cesium-137, chromium-51, cobalt-60, dysprosium-165, erbium-169, holmium-166, iron-59, phosphorus-32, potassium-42, rhenium-186, rhenium-188, samarium-153, scandium-47, selenium-75, sodium-24, strontium-89, technetium-99m, xenon-133, ytterbium-169, ytterbium-177, yttrium-90, carbon-11, nitrogen-13, oxygen-15, fluorine-18, cobalt-57, copper-64, copper-67, gallium-67, gallium-68, indium-111, iodine-123, iodine-124, krypton-81m, rubidium-81, rubidium-82, thallium-201, boron-10 and/or gadolinium-157.

In another aspect, the invention comprises a hydrogel formulation that provides sustained release of a radiation sensitizer such as doxorubicin in its free base form at the injection site. The formulation comprises dimethyl sulfoxide as a solvent and a starch polymer matrix with controlled viscosity and tissue penetration.

In another aspect, the invention comprises methods and compositions for intratumoral drug delivery in pediatric cancer patients. The therapeutic composition is administered to a brain tumor, including a medulloblastoma or ependymoma, using a sterile syringe system during or after tumor resection.

In another aspect, the invention comprises a composition suitable for simultaneous or sequential delivery of a chemotherapeutic agent and a radionuclide to a tumor region. The composition is physically stable for clinical administration and enables multimodal therapeutic action at a localized site.

In another aspect, the invention comprises a hydrogel system that produces a localized imaging signal upon administration. The imaging signal is detectable by positron emission tomography (PET), single photon emission computed tomography (SPECT), or gamma scintigraphy using a gamma- or positron-emitting radionuclide.

In another aspect, the invention comprises a composition for treating a tumor by co-administering a therapeutic agent and an imaging-capable radionuclide. The composition enables real-time or delayed imaging of drug distribution and retention.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

It should be appreciated that any patent, publication, or other disclosure material, in whole or in part, which is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The contents of U.S. Provisional Patent Application No. 63/672,066, filed on Jul. 16, 2024, and titled Starch-Based Hydrogel for Administration of Radiotherapeutic Anticancer Agents, are incorporated by reference in their entirety for all purposes, including technical disclosure, experimental data, and figures. The present application claims priority to and benefits from the subject matter disclosed therein.

### DETAILED DESCRIPTION

While various embodiments of the inventions have been shown, and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein might be employed. The various embodiments disclosed herein are combinable, as appropriate.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

Terms such as “a,” “an” and “the” are not intended to refer to only a singular entity but include the general class of which a specific example may be used for illustration. The terminology herein is used to describe specific embodiments in the present disclosure, but their usage does not delimit to the specific embodiments of the present disclosure. The term “includes” means includes but not limited to, the term “including” means including but not limited to, and the term “based on” means based at least in part on.

An immediately consecutive second feature to a first feature is devoid of another feature disposed therebetween, the features being of the same type. The feature can be a real-life feature, a calculated feature, or any other virtual feature.

When ranges are mentioned, the ranges are meant to be inclusive, unless otherwise specified. For example, a range between value 1 and value 2 is meant to be inclusive and include value 1 and value 2. The inclusive range will span any value from about value 1 to about value 2. The term “adjacent” or “adjacent to,” as used herein, includes “next to,” “adjoining,” “in contact with,” and “in proximity to.” When ranges are mentioned (e.g., between, at least, at most, and the like) the endpoint(s) of the range is/are also claimed. For example, when the range is from X to Y, the values of X and Y are also claimed. For example, when the range is at most Z, the value of Z is also claimed. For example, when the range is at least W, the value of W is also claimed.

The conjunction “and/or” as used herein in “X and/or Y”-including in the specification and claims—is meant to include the options (i) X, (ii) Y, and (iii) X and Y, as applicable. The phrase “including X, and/or Y” is meant to have the same meaning as the phrase “comprising X or Y” under currently prevailing US law.

The term “operatively coupled,” “operatively configured,” or “operatively connected” refers to a first mechanism that is coupled (or connected) to a second mechanism to allow the intended operation of the second and/or first mechanism. The coupling may comprise physical or non-physical coupling. The non-physical coupling may comprise signal-induced coupling (e.g., wireless coupling).

The phrase “is/are structured” or “is/are configured,” when modifying an article, refers to a structure of the article that can bring about the referred result. The symbol “*” designates the mathematical operation of multiplication, e.g., “times.” Fundamental length scale (abbreviated herein as “FLS”) comprises any suitable scale (e.g., dimension) of an object. For example, an FLS of an object may comprise a length, a width, a height, a diameter, a spherical equivalent diameter, a diameter of a bounding circle, a diameter equivalent of a bounding sphere.

While various portions herein may refer for simplicity to intratumoral injection or use in pediatric tumors, the present disclosure extends to any solid tumor in any clinical setting, including adult patients and non-intratumoral modes of administration where appropriate.

As described herein, the compositions, methods, and devices relate to injectable hydrogel formulations comprising a starch-based matrix, one or more therapeutic agents, and a solvent system. The therapeutic agent may comprise a chemotherapeutic agent, a radiotherapeutic agent, or a combination thereof. The hydrogel is prepared as a pre-formed injectable material, suitable for direct administration into tumor tissue.

As used herein, the term “hydrogel” refers to a crosslinked polymeric network comprising water or an aqueous solvent such as dimethyl sulfoxide. The term “starch-based hydrogel” refers to a hydrogel matrix comprising at least one polysaccharide polymer derived from starch, such as amylopectin. The term “therapeutic agent” refers to a biologically active substance intended to reduce tumor burden or disrupt tumor viability. The therapeutic agent may be a chemotherapeutic molecule, a radionuclide, or any combination thereof.

As used herein, the term “chemotherapeutic agent” may refer to a cytotoxic drug such as doxorubicin. The term “radiotherapeutic agent” may refer to a radioactive radionuclide selected for local energy emission in the form of alpha particles, beta particles, or Auger electrons. Examples include cerium-144, cerium-141, actinium-225, thorium-227, lutetium-177, and terbium-161. The therapeutic agent may be loaded into the hydrogel in a free form, unchelated form, or encapsulated form depending on the chemical compatibility with the hydrogel matrix.

In some embodiments, the term “intratumoral injection” refers to placement of the hydrogel composition directly into tumor tissue using a syringe-based or image-guided delivery approach. The term “pediatric tumor” refers to a solid tumor arising in a patient under 18 years of age, and may include tumors of the brain, spine, or soft tissues.

As used herein, the term “pre-formed hydrogel” refers to a hydrogel composition that is fully gelled prior to administration. The pre-formed hydrogel does not require in situ polymerization, crosslinking, or phase transition after injection.

As used herein, the term “dimethyl sulfoxide” (DMSO) refers to an aprotic polar solvent used to dissolve hydrophobic drugs and facilitate starch polymer solubilization. DMSO may be present at a concentration sufficient to enable hydrogel formation, typically in the range of at least about 20% v/v.

As used herein, the term “encapsulation efficiency” refers to the percentage of therapeutic agent retained in the hydrogel upon formation. Encapsulation efficiency may be determined by analytical techniques including ultraviolet-visible spectroscopy, dialysis, or mass recovery after hydrogel synthesis.

As used herein, the term “retention” refers to the percentage of therapeutic agent that remains within the hydrogel under physiological conditions over a defined time period. Retention may be evaluated by placing the hydrogel in aqueous buffer at 37° C. and sampling the release medium at defined time points.

As used herein, the term “radionuclide” refers to a radioactive nuclide capable of emitting therapeutic radiation. The radionuclide may be unchelated or chelator-free and may be directly dispersed within the hydrogel matrix without binding moieties.

As used herein, the term “imaging signal” refers to a detectable emission produced by a radionuclide incorporated into the hydrogel composition. The imaging signal comprises gamma radiation or annihilation photons resulting from a positron emission and may be visualized using nuclear medicine techniques such as single-photon emission computed tomography (SPECT), positron emission tomography (PET), or planar scintigraphy. The radionuclide may comprise a gamma-emitting or positron-emitting radionuclide such as cerium-141, lutetium-177, or actinium-225. The imaging signal enables localization, tracking, or dosimetry assessment following intratumoral injection of the composition.

As used herein, the following terms and abbreviations shall have the meanings indicated below unless the context clearly indicates otherwise:
¹³⁸Ce: Cerium-139
¹⁴¹Ce: Cerium-141
¹⁴⁴Ce: Cerium-144
²²⁴Ra: Radium-224
²²⁵Ac: Actinium-225
²²⁸Th: Thorium-228
Ce: Cerium
CeAccPro: Cerium radionuclides
CeCl₃: Cerium chloride
CPS: Counts per second
DaRT: Diffusing Alpha-Emitters Radiation Therapy
DI H₂O: Deionized water
DMSO: Dimethyl sulfoxide
DNA: Deoxyribonucleic acid
FTIR: Fourier transform infrared spectroscopy
FWHM: Full width at half maximum
H₂O: Water
HPGe: High purity germanium detector
HPLC H₂O: High-performance liquid chromatography water
IR: Infrared
LET: Linear energy transfer
MB: Methylene blue
MeOH: Methanol
NS: Not statistically significant
ROI: Region of interest
T₀: Time zero
T_{1/2}: Half-life
TRT: Targeted radionuclide therapy
UV-Vis: Ultraviolet-visible spectrophotometry
Wt %: Weight percent
γ-ray: Gamma-ray spectroscopy

The above list is provided for convenience and may not be exhaustive. Other terms may be defined contextually within the specification.

As used herein, “medical radionuclides” refers to one or more radionuclides selected from the group consisting of: actinium-225, lutetium-177, radium-223, bismuth-213, lead-212, terbium-149, fermium-225, thorium-227, thorium-226, astatine-211, astatine-217, uranium-230, iodine-131, iridium-192, iodine-125, palladium-103, cesium-131, cesium-137, chromium-51, cobalt-60, dysprosium-165, erbium-169, holmium-166, iron-59, phosphorus-32, potassium-42, rhenium-186, rhenium-188, samarium-153, scandium-47, selenium-75, sodium-24, strontium-89, technetium-99m, xenon-133, ytterbium-169, ytterbium-177, yttrium-90, zirconium-89, carbon-11, nitrogen-13, oxygen-15, fluorine-18, cobalt-57, copper-64, copper-67, gallium-67, gallium-68, indium-111, iodine-123, iodine-124, krypton-81m, rubidium-81, rubidium-82, thallium-201, boron-10, and gadolinium-157.

### Starch-Based Hyrdrogel

A starch-based hydrogel for the sustained delivery of active pharmaceutical ingredients is disclosed herein. The hydrogel comprises a dispersed phase, and a dispersion medium consisting of water, organic solvent, or a combination thereof. The dispersed phase includes a polymeric network comprising a hydrolysable polymeric substance derived from starch. In various embodiments, the polymeric starch-derived network includes amylose, amylopectin, soluble starch, native starch, or a combination thereof. In various embodiments, the polymeric starch-derived network includes a crosslinking agent which may have at least two carboxyl moieties such as a dicarboxylic acid for example d-glucaric acid.

The hydrogel is biocompatible and biodegradable, and suitable for loading with biologically active and/or active pharmaceutical ingredients ranging from small molecules therapeutics to macromolecules such as proteins and cells to radionuclides such alpha or beta emitters. Upon administration to a host animal, the hydrogel biodegrades, releasing as degradation products only naturally-occurring sugar molecules that are non-toxic.

Starch is a naturally occurring polysaccharide comprising long-chain polymers of d-glucose. Certain starch components have been contemplated for use in a biodegradable drug delivery system because of several natural advantages provided by starch including biocompatibility, biodegradability, non-toxicity, and ready availability from abundant sources. However, most hydrogels in present use, or theoretically contemplated for use as biodegradable drug delivery devices are lacking in one or more desirable properties including construction from exclusively natural products.

The disclosed composition provides a novel biodegradable starch-based hydrogel for biomedical applications. The hydrogel is designed for sustained-release delivery of biologically active ingredients and/or active pharmaceutical ingredients contained in the hydrogel. The invention incorporates several advantageous features. First, the hydrogel can be engineered to accommodate different types of therapeutic molecules ranging from small molecule to macromolecules to radionuclides. Importantly, its molecular structure can be modified to customize to meet dosing specifications of a particular application. This capability greatly expands the realm of its therapeutic uses including delivering multiple active pharmaceutical ingredients simultaneously. Envisioned uses of the hydrogel drug delivery system include inter alia cancer treatment, hormone therapy, and wound healing, but the invention is not so limited.

One preferred embodiment of a starch-based hydrogel in accordance with the present invention is a sustained release drug delivery system that provides the advantage of adjustable release kinetics, which can be specified during synthesis of the hydrogel by varying the concentrations of the polymeric components of the starting materials. More particularly, the inventive hydrogel is a starch-based biodegradable hydrogel comprising an interpenetrating network of physically entangled Starch polymer chains. In some preferred embodiments the starch-based hydrogel is cross-linked with a chemical cross-linker. During synthesis of the gel, the chemically reactive carboxyl groups of the chemical cross linker react with the-OH groups of the starch chains via condensation, creating ester linkages within the system.

Upon interaction with a biological environment, the ester linkages in the hydrogel degrade according to the same hydrolytic mechanism of the main chain backbone, resulting in the release of the chemical cross-linker while the hydrolysis of the acetal bonds of the starch results in the generation of glucose monomers, maltose dimers, and maltotriose trimers. By virtue of this design, no synthetic products are released into the body as a result of the degradation of the hydrogel. Rather, all of the breakdown products are sugars that can be safely consumed by the Surrounding cells in the tissue.

Studies described herein demonstrate that with the addition of the cross-linker, the viscosity of a starch-based hydrogel increases. With increased cross-link concentration, the degradation time of the system is extended. As a further advantage, the inventive hydrogels can be loaded with different classes and sizes of drugs, which are released from the gels over time, under physiological conditions.

Accordingly, and in one aspect, the invention provides a hydrogel for the controlled delivery of a biologically active agent. The hydrogel comprises a dispersed phase and a dispersion medium. The dispersed phase includes a polymeric network comprising a hydrolyzable polymeric Substance derived from starch, and a cross-linker. In various preferred embodiments, the polymeric starch-derived substance comprises amylose, amylopectin, or a combination thereof. In other embodiments, the hydrolyzable starch-derived substance is soluble starch. The dispersion medium consists Substantially of water.

In some preferred embodiments of the hydrogels, the cross-linker is covalently bound to the starch-derived polymeric Substance by ester linkages. Suitable cross-linkers can be molecules having at least two carboxyl moieties, including dicarboxylic acids, tricarboxylic acids, and alpha hydroxy acids.

Some embodiments of the hydrogels include a bio logically active agent, which can range from a small molecule to a macromolecule Such as a protein, polysaccharide, or nucleic acid.

Certain preferred embodiments of the hydrogels include a hydrolyzable cross-link that has the potential to cause a biological effect following the controlled release in vivo of the cross-link from the hydrogel.

The inventive starch-based hydrogels are biodegradable. Upon administration to a host animal, and following biodegradation of the hydrogel, the breakdown products that are released from the hydrogels are exclusively naturally occurring molecules and as such are non-toxic and non-immunogenic to the host animal.

The present invention provides in one aspect a hydrogel for use in biomedical applications such as drug delivery. In certain embodiments of the present invention, the colloid includes a polymeric network having both hydrophilic surfaces and hydrophobic surfaces. The hydrophilic surfaces enable the polymeric network to retain water via intermolecular forces (e.g., hydrogen bonding). The hydrophilic surfaces also increase the permeability of the polymeric network to water. In contrast, the hydrophobic surfaces reduce the water solubility of the polymeric network, thus promoting the ability of the polymeric network to interact with water (i.e., the dispersion medium) without being dis solved by it.

### Biodegradable Hydrogels Comprising Natural Polymers

Hydrogels prepared in accordance with the present invention comprise polymers that are derived exclusively from natural sources (also referred to hereinafter as “natural polymers”). As the term is used herein, a “natural polymer is meant to include a macromolecule that occurs in polymeric form in nature. Natural polymers included in the hydrogels of the invention are isolated polymers that are derived exclusively from starting materials that are natural polymers. For example, a particularly preferred natural polymer useful in the invention is starch, which is a mixture of complex carbo hydrates that is stored in abundance in e.g., seeds of plants Such as corn. Starch that is isolated from natural sources is comprised of two main polysaccharides that form natural polymers with a helical shape, i.e., amylose and amylopectin.

Hydrogels in accordance with the present invention are biodegradable. As a biodegradable drug delivery vehicle in a biological environment, natural polymers, as compared with synthetic polymers, or polymers made with a combination of natural and synthetic macromolecules, offer several advantages. Being very similar, and in Some cases identical, to endogenous macromolecular Substances, the molecules of the biological carrier can be recognized and metabolically processed by the biological environment. Thus, by incorporating only natural polymers, the hydrogels of the invention are designed to be degradable by the body into naturally occurring metabolic products, using the body's own mechanisms for breaking down these natural polymers.

This use of natural polymers provides an important advantage over drug delivery vehicles in the prior art that include synthetic polymers to Some extent. Many problems are known to be associated with the use of synthetic polymers for this purpose, including toxicity of breakdown products, stimulation of a chronic inflammatory reaction upon implantation and breakdown of the product, and lack of recognition by cells. Each of these problems is suppressed or eliminated in the hydrogels of the invention, which incorporate only natural polymers.

As a further advantage, the similarity of the natural polymers or their derivatives to naturally occurring substances introduces the interesting capability of designing biomaterials that function biologically at the molecular level, rather than at the macroscopic level.

A particularly advantageous characteristic of natural polymers is their ability to be degraded by naturally occurring enzymes, ensuring that the implant made of a natural polymer eventually will be metabolized by physiological mechanisms. Although this property may at first appear as a disadvantage since it detracts from the durability of the implant, in fact it can be used advantageously for applications in the biomaterials field in which it is desirable to deliver a specific drug on a temporary basis, following which the implant degrades completely and is disposed of by largely normal metabolic processes.

Hydrogels for biomedical applications in accordance with the present invention incorporate a polysaccharide, which is a class of biomaterials that are naturally occurring polymers. The term “polysaccharide” refers to compounds made up of many hundreds or even thousands of monosaccharide units per molecule. As in disaccharides, these units are held together by glycoside linkages, which can be broken by hydrolysis. Polysaccharides are naturally occurring polymers, which can be considered as derived from aldoses or ketoses, by polymerization with loss of water. A polysaccharide has the general formula (CHO).

By far the most abundant polysaccharides are cellulose and starch. Both are produced in plants from carbon dioxide and water by the process of photosynthesis, and both are made up of D-(+)-glucose units. Cellulose is the chief structural materials of plants, giving plants rigidity and form. Cellulose is likely the most widespread organic material on Earth.

Particularly preferred polysaccharides are starch, or starch derivatives including amylose and amylopectin. Starch makes up the reserve food Supply of plants and occurs chiefly in seeds. It is more water-soluble than cellulose, more easily hydrolyzed, and hence more readily digested (Morrison and Boyd 1983). Starch occurs as granules whose size and shape are characteristic of the plant from which the starch is CH2OH CHOH

In general, the term “starch” refers to a composite natural product comprising about 20% of a water-soluble fraction called amylose, and about 80% of a water-insoluble fraction called amylopectin. These two fractions appear to correspond to different carbohydrates of high molecular weight and formula (CH2O). Upon treatment with acid or under the influence of enzymes, the components of starch are hydrolyzed progressively to dextrin, a mixture of low molecular weight polysaccharides, (+) maltose, and finally to D-(+)-glucose. Both amylose and amylopectin are made up of D-(+)-glucose units but differ in molecular size and shape. 0053. The only disaccharide that is obtained by the hydrolysis of amylose is (+)-maltose; the only monosaccharide that is obtained is D-(+)-glucose. To account for this, it has been proposed that amylose is made up of chains of many D-(+)-glucose units, each unit being joined by an alpha-glycosidic linkage to C-4 of the next one (Morrison and Boyd 1983). Amylose, shown in Formula 1 below, is made up of long chains each containing 1000 or more D-glucose units joined together by alpha-linkages. There is little or no branching of the chain. obtained. When intact, starch granules are insoluble in cold water. If the outer membrane has been broken, for example by grinding, the granules swell in cold water and form a gel. When the intact granule is treated with warm water, a soluble portion of the starch diffuses through the granule wall. In hot water, starch granules Swell to Such an extent that they can burst.

Polysaccharide mixtures occur naturally, and it has been recognized that binary carbohydrate gels can be used as models for complex cellular structures involving the recognition step in certain host-pathogen interactions (see, for example, Dumitriu 1996).

Amylopectin, like amylose, is made up of chains of D-glucose units, with each unit being joined by an alpha glycoside linkage to C-4 of the next D-glucose unit. However, the structure of amylopectin is more complex than that of amylose. Molecular weight determination of amylopectin using physical methods as described, shows that there are up to a million D-glucose units per molecule of amylopectin. This molecule has a highly branched structure, consisting of several hundred short chains of about 20-25 D-glucose units each. Native, or natural, form, starch occurs as a granule made up of several alternating layers having crystalline and amorphous forms. The regions of long-range crystallinity appear to involve crystallization of the amylopectin component of starch, whereas the amylose component represents the amorphous phase of starch granules (Dumitriu 1996).

Amylose is a polysaccharide composed of unbranched chains of D-glucose units joined by C-1,4′-glycosidic linkages. The structural characteristics of amylose gels (2%- 8% w/vol) at different scales of organization have been studied by electron microscopy, mild acid hydrolysis, differential scanning calorimetry, and size-exclusion chromatography. Amylose gels have been shown to exhibit a macro porous structure, with a mesh size of 100-1000 nm, containing filaments 20+10 nm wide. These filaments result from the association of segments of amylose chains that are oriented obliquely to the filament axis. These amylose fragments are partially organized in a B-type crystalline array. Upon acid or enzymatic treatments, the amorphous segments of amylose chains are hydrolyzed (Dumitriu 1996). 0057. In contrast to amylose, amylopectin is a branched polysaccharide. Like amylose, amylopectin is composed of chains of D-glucose units joined by C-1,4-glycosidic link ages. Unlike amylose, amylopectin also contains C-1,8-glycosidic linkages, which create branches in the polysaccharide. Physical techniques such as x-ray diffraction (XRD), Small angle neutron Scattering (SANS), Small angle X-ray scattering (SAXS), and electron microscopy (EM) have provided information on the nature of amylopectin crystallites. Such studies have shown a periodicity in native starch occurring at 10 nm, which is thought to be the repeat distance between the amorphous and crystalline regions (Dumitriu 1996). Amylopectin is a high molecular weight natural polymer with non-random branches. The branching of the polymer is extensive and is responsible for the molecule's large hydrodynamic Volume and ability to gel at concentrations of 3% and above in aqueous solution. The branching is known to be non-random; however, there is disagreement as to the precise architecture of the molecule because of the influence of Steric hindrance and the placement of inner and outer chains. One widely accepted theoretical model of the molecular structure of amylopectin proposed by Durrani and Donald (1995) depicts a cascade-type branching structure, with the amylopectin branches arranging themselves in clusters of tiered branches.

In some embodiments of the present invention, the starch-derived polymeric network comprises or is derived from soluble starch. As discussed above, “soluble starch” refers to starch molecules that have been degraded. Soluble starch is derived from naturally occurring starch, for example, by acid hydrolysis with hydrochloric acid, resulting in a product having a molecular weight of about 300 g/mole. As mentioned, amylopectin and soluble of starch are both long chain molecules composed of glucose monomers; however, they differ greatly in size. In contrast to Soluble starch, the molecular weight of amylopectin can be as high as 10 g/mole. Starch-based hydrogels comprising polymer networks made from soluble starch as a starting material are prepared in same way as those made from amylose and/or amylopectin.

A plurality of different three-dimensional structures can be formed when two polysaccharides are mixed together and gelled to form a hydrogel in accordance with the instant invention. The chemical structures of various embodiments of the inventive hydrogels are dependent on the nature of the starting components, the rate and extent of de-mixing, and the method or mechanism of gelation.

The simplest type of structure is obtained when a first component forms a network, and a second component is merely contained within the first component. In more complex hydrogels in accordance with the invention, at least three types of gel structure can occur if both polysaccharides contribute to the network. In one type, the gel is an interpenetrating network in which both polysaccharides associate independently to form separate networks but interlace with each other. In a second structural variation, a phase-separated network results if some degree of de-mixing occurs prior to gelation, and the two networks are spatially separated. In yet a third type of polysaccharide hydrogel structure, a coupled network is formed if the two networks chemically bond to each other during gelation.

One preferred embodiment of a naturally derived polymer hydrogel Suitable for drug delivery in accordance with the present invention is a hydrogel-based drug delivery system comprising starch, as described above. Based on study of the properties and characteristics of such hydrogels, it is believed that as the starch chains are heated, dispersed and stirred to prevent sedimentation during synthesis of the gels, the randomly coiled Starch chains uncoil and become hydrolyzed, then recoil upon cooling, creating physical entanglements among neighboring chains. Also, as the dispersed starch chains are heated in Solution, the cross-linker binds to the main chain backbone.

Gelation, as the term is used generally herein, refers to the cross-linking of a plurality of polymer chains into progressively larger branched polymers, up to and including a single molecule spanning an entire system. “Starch gelation” as the term is used herein, refers to a process by which solubilization, hydration, and Swelling of Starch molecules occurs. For example, starch can be heated in a solution, during which it undergoes dispersion, randomly coiled chains uncoil and become hydrolyzed, and then recoil upon cooling, creating physical entanglements among neighboring chains. See further description, for example, in Whistler (1984). Starch gelation can also occur by hydrogen bonding. Next to physical entanglement, hydrogen bonding is the second most important mechanism of starch gelation. The solvent properties of water are known to be responsible for the hydrogen bonding of the starch gels (Morawetz, 1965). Each oxygen molecule is Surrounded by four hydrogen atoms in a tetrahedral array. With two of the hydrogen molecules, the oxygen molecule forms a covalent bond; two hydrogen molecules belonging to neighboring water molecules, the oxygen molecules form a hydrogen bond. A hydrogen bond can form between two neighboring water molecules only if a hydrogen molecule lies close to the line connecting the oxygen atoms. This constraint prevents the starch molecules from packing as efficiently as they would if their interactions were solely due to dispersion forces and dipole-dipole interactions. During the gelation of starch in water, hydrogen bonding occurs around the perimeter of the starch helices, as well as the within the alpha helix. The alpha helix is capable of accommodating as many as 32 interstitial water molecules per turn of the alpha helix.

The phenomenon of “polymer immiscibility” is well known in the art of polymer chemistry and arises as a result of the generally unfavorable interactions between polymer species. Even a small positive free energy of interaction between different polymer species can result in limited miscibility due to the small entropy gain on mixing these high molecular weight species. The miscibility of polymers in Solution decreases with increasing polymer concentration and is rare at high concentrations. If the segregation factor is strong, de-mixing is predicted when the polymer chains start to become entangled above the coil-overlap threshold. The phase behavior of ternary systems (i.e., polymerl+polymer 2+solvent) can be strongly affected by polymer-solvent interactions. In general, immiscibility is increased when the affinity of one polymer for the solvent is significantly different from that of the other. Like synthetic polymers, biopolymers also exhibit immiscibility, with perhaps the best known example being the gelatin-gum Arabic-water system. Compared to synthetic polymer mixtures, there is relatively little information on the phase behavior of polysaccharide mixtures. In general, lower molecular weight species appear to have more affinity for the solvent.

As discussed, starch occurs naturally in seeds as a mixture of amylose and amylopectin molecules which are organized into a semi-crystalline granule. The behavior of aqueous solutions of starch is of interest because starch is processed by heating in the presence of water. Whereas amy lose is essentially a (1-4) alpha-D-glucan, amylopectin is a (1-4) alpha-D-glucan with an average of one in every 20-25 residues branched at position 6. Because these polymers are chemically quite similar, it might be expected that immiscibility would only be observed at very high concentrations. However, it has been found that even moderately concentrated aqueous solutions of amylose and amylopectin exhibit immiscibility (Kalichevsky 1987).

According to Kalichevsky's theoretical study of mixtures of amylose and amylopectin of varying concentrations from pure amylose to pure amylopectin in a 10% total aqueous solution, the binodal phase diagram is not symmetric. Rather, it is shifted towards the amylose-rich phase. This is consistent with the behavior predicted for a mixture containing polymers of unequal molecular weight, the bimodal being displaced towards the polymer of lower molecular weight. As discussed by this author, the tie lines between phases at equilibrium with each other slope up to the amylose rich phase, indicating that this phase has a higher affinity for the water solvent.

The investigation of the phase behavior of amylose and amylopectin, using amylose of different molecular weights and structures as well as different temperatures (70 C-90 C), shows that these factors do not strongly affect the unfavorable interaction between these polysaccharides which gives rise to the phase separation. It is noted that the differences in molecular weight between the amylose samples are small, relative to the large differences between amylose and amylopectin. From the observation that phase separation only occurs at concentrations well above C*, (defined as minimum polymer concentration to induce phase separation), it is apparent that the segregation factor is not very strong. It has been observed that immiscibility becomes greater with increasing molecular weight, so the high molecular weights of these polysaccharides (especially the amylopectin), encourage phase separation.

The incompatibility of the linear and branched polymers of starch in aqueous solution has important implications. Incompatibility may also affect the types of interactions which can occur and should be considered as part of the gelatinization behavior of starch on heating in water (Kalichevsky 1987).

The ratio of the polysaccharides (e.g., of amylose to amylopectin) in a starch derived hydrogel can influence the properties of the polymeric network. Accordingly, the performance of the hydrogel can be altered by changing the ratio of the polysaccharides. For example, the susceptibility of the hydrogel to hydrolytic or enzymatic degradation can be altered by adjusting the amylase: amylopectin ratio in an exemplary hydrogel synthesized from these two starch components as starting materials.

A factor affecting the performance of a hydrogel in accordance with the present invention is the degree of cross linking that exists within the polymeric network. A “cross link, as the term is used herein, refers to a crosswise connecting part (as an atom or group) that connects roughly parallel chains in a complex chemical molecule (e.g., a polymer). In the majority of instances, a cross-link is a covalent structure, but the term is also used to describe sites of other chemical interactions (e.g., ionic interactions), portions of crystallites, and even physical entanglements.

The process of creating cross-linking in a starch derived polymeric network is generally known in the art and typically involve the use of a “cross-linker, also referred to as a “cross-linking agent” or a “cross-linker molecule.′ “Chemical cross-linking is defined as a linking mediated by the reaction of a linear, or branched polymer with at least one cross-linking agent of relatively small molecular weight. As discussed, the function of the cross-linking agent is to link two larger molecular weight chains through its di- or multifunctional reactive chemical groups (Ratner 2004). The cross-linking agents can be added to the polymer Solution after the polymer has been produced, the reactive species of the agents reacting with the polymer chains to form a chemically cross-linked system.

“Copolymerization cross-linking refers to a reaction between a solution of one or more types of monomers including one multi-functional monomer that is present in relatively small quantities, where the polymerization of the polymer and the chemical cross-linking of the system occurs in one step. Another method involves combining monomer and linear polymeric chains that are cross-linked by means of an interlinking agent (Ratner 2004).

Cross-linking is a well-established method for chemical modification of polymers. Varying degrees of cross linking can be introduced into polysaccharides depending on the purpose, e.g., to generate larger molecular aggregates with enhanced viscosity profiles, or to enable preparation of insoluble products with a wide range of Swelling characteristics (Dumitriu 1996) Some common cross-linking agents include bi- and tri-functional reagents, such as epichlorohydrin, bisepoxides, dihalogenated reagents, glutaraldehyde, acetaldehyde, formaldehyde, maleic and oxalic acid, dim ethylurea, polyacrolein, diisocyanates, divinyl Sulfate, ceric redox systems, and S-triazine.

There are several factors to consider when determining appropriate conditions for chemically cross-linking a starch-based hydrogel in accordance with the present invention. For example, the extent of the reaction and the number and character of side reactions must be considered. An important parameter that can be used for identification of the final cross-linked structure is the cross-linking ratio (CR), which is defined as the ratio of the moles of cross-linking agent to the moles of polymer repeating units (Dumitriu 1996). For bi functional cross-linking agents, the number average molecular weight between cross-links, Mc, may be determined by the relation M. where M is the molecular weight of the repeating unit.

It is generally appreciated that the addition of a chemical cross-linker can be beneficial for improving both the integrity of a hydrogel and the predictability of its mechanical properties. One important and recognized aspect of biopolymer gelation is the relationship between gel modulus and concentration. This relationship tests network theories which relate the molecular structure of the gel to the most apparent macroscopic aspect of the gel, i.e. its mechanical properties (Durrani and Donald 1995).

Various methods can be utilized in making a hydrogel that comprises a cross-linked Starch-derived polymeric network. For example, in one method, a mixture including a starch-derived polymeric network and water is heated to at least partially solubilize the starch-derived network. During heating, this polymeric network, which for example may include amylose and amylopectin, partially unravels. The heating also encourages the helical regions of the polymeric network to uncoil. This unraveling and uncoiling of the polymeric network allow the water in the mixture to more readily permeate and solvate the network. Additionally, some of the glycosidic linkages in the network are hydrolyzed, further encouraging the unraveling/uncoiling and, hence, the solvation of the network.

As the solvation processes continue, the network is dispersed in the water, thereby increasing the homogeneity of the mixture. After the homogeneity has increased sufficiently, a cross-linking agent is added to the mixture. The heating of the mixture continues, encouraging the cross-linking agent to bond to the polymeric network, to yield a cross-linked starch derived polymeric network.

The temperature(s) attained at this stage of heating may vary, depending, inter alia, on several factors including the identity of the cross-linking agent; the concentration of the cross-linking agent in the mixture; and the desired degree of cross-linking in the polymeric network. Thereafter, the mixture is cooled, encouraging the polymer chains in the network to entangle and coil. At least some of the water present in the network upon commencement of the cooling is retained therein, leading to the formation of the hydrogel.

In some preferred embodiments of cross-linked hydrogels in accordance with the invention, the linkage occurs by ester linkages. Ester linkages are preferred for this purpose because esters degrade hydrolytically, giving the gel its, biodegradable properties. Typically, ester linkages are formed from the condensation reactions of alcohols and carboxyls. For example, in some preferred embodiments of the inventive hydrogels, the cross-linker molecules utilize alcohol groups present in starch derivatives such as amylose and amylopectin.

The range of molecules that can be effectively utilized as cross-linkers in a hydrogel of the present invention is not particularly limited. Any compound having at least two functional groups (carboxyl moieties) can be used as a chemical cross-linker to react, e.g., with the alcohol groups of the starch derivatives. Cross-linkers suitable for use in the present invention include but are not limited to: dicarboxylic acids (e.g., Oxalic acid, malonic acid. Succinic acid, glutaric acid, adipic acid, pimelic acid, Suberic acid, azelaic acid, sebacic acid, pthalic acid, isophthalic acid, and terephthalic acid; tricarboxylic acids (e.g., citric acid, isocitric acid, aconitic acid, and propane-1,2,3-tricarboxylic acid); and alpha hydroxyl acids, e.g., tartaric acid.

In some of preferred embodiments, the hydrogel comprises a starch-derived polymeric network that is covalently cross-linked using D-glucaric acid lactone (e.g., D-glucaro-1,4-lactone) as a cross-linker. For example, the carboxyl group of D-glucaro-1,4-lactone that includes the C-6 carbon can react with a primary hydroxyl group on a first polymer chain of amylose or amylopectin via an esterification reaction, thereby resulting in a covalent bond between the two. Additionally, the pendant lactone can react with a primary hydroxyl group on a second polymer chain of amylose or amylopectin via a transesterification reaction, resulting in a covalent bond between these two, thereby completing the cross-link.

It is noteworthy that ester linkages are susceptible to hydrolysis, as are the glycosidic linkages in amylose, amylopectin, and other polysaccharides. Accordingly, a hydrogel that comprises a starch-derived polymeric network that is cross-linked via an esterification process is “biodegradable.” meaning that the hydrogel will erode or degrade in Vivo, essentially yielding only biocompatible Substances, including the cross-linker molecule.

It will be readily apparent to those of skill in the art of polymer production that this process can also be used to synthesize a starch-derived polymeric network that is cross linked with any of the above-described cross-linkers.

Methods for testing the effects of cross-linkers on starch gel characteristics are known and have been described, for example, in studies of a product known as Cross-linked High Amylose Starch (CLHAS). CLHAS was introduced into the market several years ago as the controlled release device, Contramid R. (Labopharm Inc, Laval, Quebec, Canada). Contramid (R) is a starch-based gel of made of amylose as the starch component, cross-linked with epichlorohydrin. Unlike the hydrogels of the instant invention, this product, which can be administered orally or implanted Subcutaneously, is designed to undergo gelation in Vivo, i.e. only after it is placed in a biological environment. When placed in Solution, the product Swells to form an elastic gel. As mentioned, the native starch granule is heterogeneous both chemically (comprising both amylose and amylopectin) and physically (having both crystalline and amorphous regions). The presence or absence of crystalline order is often a basic underlying property of starch. Depending on their origins, various types of native starches present specific morphologies, giving distinctive X-ray powder patterns termed types “A.” “B,” or “C” polymorphs. The sharpness of the X-ray diffraction pattern of starch granules depends on their water content, with the B-type being more sensitive to hydration than the A-type starch. The role of crystallinity in release control has been studied in a series of powders, tab lets, and films of high amylose starch having varying degrees of cross-linking (Ispas-Szabo et al.).

In certain preferred embodiments of the invention, the cross-linker in the hydrogel is in itself a biologically active agent. As a non-limiting example, there is a significant body of evidence tending to establish that D-glucaro-1,4-lactone is a biologically active agent. It has been Suggested, e.g., that D-glucaro-1,4-lactone possesses anti-cancer activity, among other beneficial biomedical effects (see, e.g., Walaszek, 1990). Certain preferred embodiments of hydrogels in accordance with the present invention comprise a starch-derived polymeric network that is cross-linked with D-glucaric acid (e.g., D-glucaro-1,4-lactone) and accordingly may have application for the treatment of cancer. 0089) Hydrogels prepared in accordance with the present invention are Suitable for parenteral administration to a warm-blooded organism, preferably a mammal, and most preferably a human. In one particularly preferred embodiment, the hydrogel is formulated as an injectable gel. Upon administration, (e.g., intravenously), the hydrogel can be used to increase the local concentration of a drug or therapeutic agent in the bloodstream. Other preferred embodiments can be administered parenterally by other delivery routes and can facilitate the selective targeting of a location in the organism such that, at least initially, the concentration of the drug in that location can be increased substantially, relative to the overall concentration of the drug or therapeutic agent in the organism. Thus, as one example, a hydrogel comprising a starch-derived polymeric network that is loaded with an anticancer drug can be administered locally, e.g., by injection, to a location in proximity to a tumor requiring treatment.

In certain preferred embodiments of the present invention, the hydrogel comprises a starch-derived polymeric network that is cross-linked with a biologically active form of D-glucaric acid (e.g., D-glucaro-1,4-lactone). The cross link in this instance can serve at least two purposes. In addition to causing a biological effect upon release from the hydrogel as described above, such a cross-link can serve to alter the degradation properties of the hydrogel. Accordingly, the degree of cross-linking affects the release kinetics of the biologically active form of D-glucaric acid. A Suitable degree of cross-linking can, for example, increase the reliability of the performance of the hydrogel, insofar as release kinetics are concerned. It is worth noting that, in this instance, the degree of cross-linking acquires even greater significance because it is directly related to dosage.

Other factors affecting release kinetics from the hydrogel include: the ratio of amylose to amylopectin; and the density of the hydrogel. Suitable formulations can be determined by one skilled in the art and will vary depending, interalia, on the route of administration; the desired biological effect (e.g., anti-cancer activity); and the desired release kinetics (e.g., Sustained release and other types of controlled release). The particular formulations also may vary, depending on whether the desired biological effect is therapeutic, prophylactic, or diagnostic.

By controlling the degree of cross-linking during synthesis, it is possible to achieve distinct degradation properties and drug release profiles. During synthesis, specific concentrations of starch are added to aqueous Solutions of the chemical cross-linker. The gel network is then formed as the pre-polymer Solution is heated. Upon heating, the helices of the polymer chains open up and entangle with neighboring chains, forming aggregates. The starch components are immiscible in solution due to differences in solubility parameters, as governed by the various molecular weights of the chains present. This phenomenon induces phase separation within the system. Accordingly, like chains physically entangle with each other during the process and an interpenetrating network of the high and low molecular weight chains is formed. At the same time, the carboxyl groups of the chemical cross-linker react with the alcohol groups of the starch via condensation, creating ester linkages that covalently tie the network together.

The degree of cross-linking within the system determines the pore size of the network, a factor that is important with respect to release of therapeutic agent from within the system. By increasing or decreasing both the physical and/or chemical crosslink density of the polymer, the pore size of the network can be altered in a predictable and reproducible way.

Studies to characterize the polymeric nature of starch-based hydrogels of the invention are further described in the Examples below and demonstrate that a plurality of starch-based cross-linked biodegradable gels can be synthesized using methods described herein. RAMAN spectroscopic analysis has confirmed the presence of ester linkages in the starch-based hydrogels, which were due to the addition of a chemical cross-linker during synthesis of the gels. Polymer rheology studies have demonstrated that the viscosity of the hydrogels increases with increasing concentration of cross-linker. As further described below, it has also been determined that therapeutic agents can be loaded into the hydrogels (either bound by covalent bonds or otherwise associated with or incorporated into the gels by non-covalent forces), and that therapeutic agents (ranging from small molecules to macromolecules such as polypeptides) are released from the gels over time.

### Starch-based Hydrogels Comprising Biologically Active Agents.

In some embodiments of the present invention, the dispersed phase (colloid) of the hydrogel includes at least three different chemical substances: a starch-derived polymeric network; a cross-linking agent; and a biologically active agent.

The starch-derived polymeric network is cross linked to facilitate the controlled release in vivo of the bio logically active agent. In at least Some of these embodiments, the starch-derived polymeric network is cross-linked also to facilitate the retention in vitro of a biologically active agent. The biologically active agent can complex with the starch derived hydrogel by one of several mechanisms. The biologically active agent can be incorporated into the matrix of the gel network. Alternatively, the biologically active agent can complex within the molecular structure of the individual polymer chains comprising the network. The biologically active agent can also react with the main chain backbone of the polymers comprising the network. The biologically active agent can be loaded into the hydrogel either during the Syn thesis of the network, or after the network has formed, depending upon the type of agent being loaded. For instance, a protein-like biological agent would not be able to withstand the heat required to form the hydrogel network, so it can be added after the gel has been formed.

To load the gel after synthesis, a simple diffusion method can be used to dissolve the biologically active agent in PBS and add the gel to the solution. The active agent then diffuses into the gel and becomes complexed within the gel matrix.

The cross-link can change the pore size of the matrix, thereby impeding diffusion of the biologically active agent out of the gel matrix. The cross-link can in some instances change the mechanical properties of the starch derived polymeric network, thereby enhancing the ability of the network to retain biologically active agents. Such changes can affect the helical regions of the starch-derived polymeric network, allowing these regions to more readily accept and retain a biologically active agent (e.g., an antibiotic, or a drug to be delivered to a particular site in the body, for example). It is worth noting that in embodiments in which the biologically active agent is complexed with the helical or other regions of the starch-derived polymeric network, diffusion can be a significant factor in effecting the release of the biologically active agent from the hydrogel. Thus, one skilled in the art will consider the process of diffusion as well as the degradability of the hydrogel when formulating and designing Such a hydrogel.

Biocompatibility of hydrogels in accordance with the present invention can further include other natural polymers such as lipids, proteoglycans, or polysaccharides. One of the most advantageous features of natural polymers in this regard is the ability of these polymers to be degraded by naturally occurring enzymes and metabolized by normal physiological mechanisms (Dumitriu S, Polysaccharides in Medical Applications, Marcel Dekker Inc., New York, N.Y., 1996).

The invention relates to the use of biodegradable carbohydrate-based hydrogels as delivery systems for concurrent delivery of more than one therapeutic agent for biomedical applications including, but not limited to delivery systems for whole, viable biological cells.

For example, low drug penetration and drug resistance complicates treatment of solid tumors. These problems are increased by the presence of multiple cell types in solid tumors. The presence of multiple cell types creates a need for delivery of more than one therapeutic compound via single delivery mechanism. This concurrent, combinatorial therapy has been shown to increase efficacy of chemotherapy. (Bae et al, J. Cont. Rls., 122 (2007) 324-330; Hirsch et al, Cancer Res., 69 (2009); (19) 7507-11: Bouhadir et al, Biomaterials 22 (2001); 2625-2633). Not only is simultaneous release important for eradication of tumors, but also the advantage of a gel system is that the hydrogel can be injected locally at a Solid tumor site to increase chemotherapy concentration at the target tissue and decrease drug concentrations at non target tissues preventing drug toxicity to healthy tissues as well as decreasing side effects.

Concurrent delivery of multiple therapeutic compounds, with different mechanisms of action, has been found to create synergistic effects in treating cancer and preventing metastasis. Id. This synergistic effect not only leads to more efficacious treatment, but allows for lower-dose treatments, thereby lowering toxicity to non-cancerous tissues.

In order to more effectively treat multiple cell types, take advantage of synergistic drug interactions, and allow for lower dose treatments, methods of concurrent delivery of multiple therapeutic agents is desirable.

Hydrogel composition of a kind of physical crosslinking and its preparation method and application.

The invention belongs to a macromolecular material and medical technical field, related to hydrogel composition of a kind of physical crosslinking and preparation method thereof.

The compositions of the present disclosure have significant anticancer activity against cancer cells. Intratumoral administration of the compositions results in effective growth inhibition of cancer cells. The tumor inhibition rate of the composition was significantly higher than that of the anticancer agent alone. The injectable hydrogels of the present invention are drug delivery systems that can increase the efficacy of cancer chemotherapy.

Compositions according to the present disclosure effectively deliver active agents in an individual in a sustained manner. In addition, the active agent is administered to the subcutaneous area, tumor site, peritumoral site, or the resulting cavity after tumor resection.

In another aspect, compositions according to the present disclosure eliminate the need for surgery and provide the ability to form any desired implant shape.

In another aspect, the compositions according to the present disclosure of the described above further comprises the step of administering the hydrogel to the individual wherein the step is selected from the group consisting of intratumoral injections, peritumoral injection, injection of excised tumor Lumen, subcutaneous injection, oral deliver, ocular delivery, transdermal, ophthalmic, wound healing, intraperitoneal injection, gene delivery, tissue engineering, colon-specific drug delivery.

Compositions according to the present disclosure are readily injected subcutaneously, intramuscularly, intratumorally, peritumorally, or into the cavity resulting from tumor resection.

The present invention relates to a biodegradable drug delivery composition comprising starch and a pharmaceutically active ingredient. Also described are methods for preparing such starch-based compositions using organic solvents.

Drug delivery systems have been used to deliver a variety of drugs, which are generally formulated to deliver specific drugs, whether hydrophobic or hydrophilic. Depending on drug solubility, the drug formulations differ in concentration of the polymer in the formulation, the type of polymer used in the formulation, the molecular weight of the polymer, and the solvent used in the formulation.

Drug delivery compositions that are injectable biodegradable and are injected into the body. The biodegradable drug composition of the present invention comprises a starch-based hydrogel serves as a reservoir for an active pharmaceutical ingredient.

In addition, the biodegradable drug delivery composition of the present invention may be a sustained release formulation which extends the rate of release over time.

The present invention provides a biodegradable drug delivery composition comprising a starch-based hydrogel comprising a 3 dimensional polymeric network, a solvent comprised of aqueous or organic solvent where the aqueous can be phosphate buffered saline and the organic solvent can be DMSO, and one or more pharmaceutically active ingredients including but not limited to a radionuclide or radionuclide, radiation sensitizer, or chemotherapy agent.

One or more pharmaceutically active ingredients are entrapped in the hydrogel network drug delivery compositions. Representative drugs and biologically active agents used in the present invention include peptide drugs, protein drugs, desensitizing agents, radiation sensitizing agents, antigens, vaccines, vaccine antigens, anti-infective agents, antibiotics, antimicrobial agents, antidiabetic agents, steroidal anti-inflammatory agents, decongestants, pupils reducing agents, anticholerniergic, sympathetic stimulants, sedatives, hypnotics, pschostilmulants, stabiliziers, androgenic steroids, estrogens, progesterone preparations, humoral preparations, prostaglandins, analgesics, corticosteroids, antispasmodics, antimalarials, antihistimines, cardiac agents, non-steroidal anti-inflammatory agents, anti-Parkinson's agents, antihypertensive agents, beta-andrenergic blockers, nutrients, gonadotropin-releasing hormone agonists, chemotherapeutic agents, radionuclides, and radionuclides.

Thus, combinations of drugs may also be used in the biodegradable drug delivery compositions of the present invention. For example, the chemotherapy drug doxorubicin that is also a radiation sensitizing agent with an alpha therapy radionuclide such as actinium-225.

The active ingredient may be released for a period of days to a year a more, depending on the type of treatment required and the biodegradable drug delivery composition used.

The biodegradable drug delivery composition may further comprise a pharmaceutically acceptable carrier, adjuvant, or vehicle. Acceptable carriers can be salts, buffered salts, nd the like. After formulation of the drug with the hydrogel a carrier may be added to the biodegradable drug delivery composition.

Adjuvant may be formulated at the same time when admixing the drug. Adjuvants that can be used in this regard are alum, aluminum phosphate, calcium phosphate, MPL, CpG motifs, modified toxins, saponins, endogenous stimulatory adjuvants such as cytokines, Freund's complete and incomplete adjuvant, ISCOM Type adjuvants, muramyl peptides, and the like.

The vehicle can be any diluent, additional solvent, filler or binder that can alter the delivery of the active ingredient when need in a biodegradable hydrogel drug delivery composition. The vehicle may be immunogenic to enhance chemotherapy effects.

In another aspect, methods of producing a composition according to the formula are provided.

In another aspect, the present technology also provides compositions (pharmaceutical compositions and medicaments comprising any of one of the embodiments of the compounds disclosed herein and a pharmaceutically acceptable carrier or one or more excipients or fillers.

In another aspect, the present technology provides a method of treating cancer/disease by administering an effective amount of the targeting composition according to a subject having cancer/disease.

Pharmaceutically acceptable forms of compounds described herein are within the scope of the present technology and include acid or base addition or salts which retain the desired pharmacological activity. When the compound of the present technology can be formed with water, buffers, or organic solvents.

Although targeted radiotherapy has been practiced for some time using the systemic administration of radionuclides, the formulation of those radionuclides especially of larger size provide insufficient stability. These radio nuclides include actinium, radium, bismuth, and lead radionuclides.

The present technology provides new complexes that are substantially more stable than those of the conventional art and thus these new complexes can advantageously target cancer cells more effectively, with substantially less toxicity and non-targeted tissue that complexes of the art.

The present technology can be formulated with radionuclides of different energies including but not limited to alpha emitters, beta emitters, gamma emitters, and species for neutron capture.

The pharmaceutical compositions may be prepared by mixing one or more compounds of the formulas xx with active pharmaceutical ingredients including radionuclides with pharmaceutically acceptable carriers, excipients, binders, diluents or the like to prevent and treat diseases such as cancer. The compounds and compositions described herein may be used to prepare formulations and medicaments that treat solid tumors such as brain tumors, melanoma, bone, prostate, breast, bladder, lung, liver, pancreatic, kidney, lymphoma, thyroid, head and neck, ovarian, uterine, testicular, colon, rectal, and penile.

Such compositions may be in the form of for example granules, powders, tablets, capsules, syrup, suppositories, injections, emulsions, elixirs, suspensions, or solutions. The instant compositions may be formulated for various routes of administration for example intratumoral, intrathecal, intracavitary, oral, parenteral, topical, rectal, nasal, vaginal administration, or via implanted reservoir. Administration includes but is not limited to subcutaneous, intravenous, intraperitoneal, and intramuscular. The following dosage forms are given by way of example and should not be construed as limited the instant present technology.

Specific dosages may be adjusted depending on the conditions of disease, the age, body weight, general health conditions, sex, and diet of the subject, dose intervals, routes of administration, excretion rate, and combinations of drugs. Any of the above dosage forms containing effective amount are well within the bounds of routine experimentation and therefore, well within the scope of the instant present technology.

Various assays and model systems can be readily employed to determine the therapeutic effectiveness of the treatment according to the present technology.

For the indicated condition, test subjects will exhibit a reduction in tumor burden or one or more symptoms caused by or associated with the disorder in the subject compared to placebo-treated or other suitable control subject.

In another aspect, the present technology provides a method of treating cancer by administering an effective amount of the targeting composition to a subject having cancer. Since a cancer cell targeting agent can be selected to target an of a wide variety of cancers, the cancer considered herein for treatment is not limited. The cancer can be essentially any type of cancer.

In some embodiments, the composition may be administered locally, at the site where the target cells are present including specific tissue, organ, fluid including cerebrospinal fluid, etc. Some examples of applicable body tissues include breast, lunch, stomach, intestines, prostate, ovaries, cervix, pancreas, kidney, liver, skin, lymph nodes, bones, bladder, uterus, colon, rectum, and brain. The cancer can also include the presence of one or more carcinomas, sarcomas, lymphomas, blastomas, or teratomas. In some embodiments, the cancer is pediatric brain tumors including medulloblastoma.

In accordance with the present disclosure, the long half-life of actinium-225 (225Ac3+) necessitates its stable complex retention in vivo to reduce off-target damage to normal tissues arising from the release of free 225Ac3+.

The compositions of claim 13 where the selected radionuclide include but are not limited to actinium-225, radium-223, bismuth-213, lead-212, terbium-149, fermium-225, thorium-227, thorium-226, astatine-211, astatine-217, and uranium-230.

The composition including selective cancer cell targeting groups containing amino acids linked by peptide bonds or cancer-targeting antibody or antibody fragment.

The present disclosure provided for a method of treatment cancer in a subject, the method comprising administering to a subject having cancer an effective amount of a composition or a pharmaceutically acceptable form of an alpha-emitting radionuclide, beta-emitting radionuclide, gamma emitting radionuclide, or radionuclide for neutron capture.

In accordance with the present disclosure, the hydrogel composition may be formulated for encapsulation, retention, and/or localized delivery of a wide range of radionuclides for diagnostic and therapeutic purposes. The radionuclide may comprise an alpha emitter, a beta emitter, a gamma emitter, an Auger electron emitter, and/or a neutron capture radionuclide. The radionuclide may be selected to induce therapeutic effects such as tumor ablation, targeted radiocytotoxicity, or palliative relief of metastatic symptoms.

Reactor-derived radionuclides suitable for encapsulation may comprise one or more of actinium-225, bismuth-213, caesium-131, caesium-137, chromium-51, cobalt-60, dysprosium-165, erbium-169, gold-198, holmium-166, iodine-125, iodine-131, iridium-192, iron-59, lead-212, lutetium-177, palladium-103, phosphorus-32, potassium-42, radium-223, rhenium-186, rhenium-188, samarium-153, scandium-47, selenium-75, sodium-24, strontium-89, technetium-99m, thorium-227, xenon-133, ytterbium-169, ytterbium-177, and/or yttrium-90.

Cyclotron-derived radionuclides suitable for hydrogel incorporation may comprise one or more of astatine-211, bismuth-213, carbon-11, cobalt-57, copper-64, copper-67, fluorine-18, gallium-67, gallium-68, indium-111, iodine-123, iodine-124, krypton-81m, nitrogen-13, oxygen-15, rubidium-81, rubidium-82, strontium-82, thallium-201, and/or actinium-225.

Radionuclides useful for neutron capture therapy may comprise boron-10, gadolinium-157, and/or ytterbium-176. In some embodiments, the composition comprises a parent radionuclide such as molybdenum-99 used for generator-based production of technetium-99m. The encapsulated radionuclide may be selected based at least in part on emission profile, tissue penetration characteristics, half-life, or compatibility with existing imaging modalities.

The disclosed hydrogel system may be formulated to support encapsulation of one or more of these radionuclides for use in palliative treatment (e.g., strontium-89 or samarium-153 for bone pain), tumor-directed ablation (e.g., lutetium-177 for neuroendocrine tumors), diagnostic imaging (e.g., technetium-99m or indium-111), or emerging modalities such as boron neutron capture therapy. The encapsulation may be chelator-free or may optionally employ a chelating agent depending on radionuclide chemistry.

The use of radionuclides in cancer therapy is important and growing. Cancerous growths, as well as other diseased cells are sensitive to damage by radiation. For this reason some cancerous growths can be controlled or eliminated by irradiating the area containing the growth. Internal radionuclide therapy is administered by planting a small radiation source usually an alpha, gamma, beta emitter, or source for neutron capture in the target area, ie solid tumor. iodine-131, iridium-192, iodine-125, palladium-103, IR-192.

In some embodiments, the composition is a pre-formed hydrogel. The hydrogel may comprise a polymeric matrix derived from starch, a solvent comprising dimethyl sulfoxide, a therapeutic agent comprising a chemotherapeutic compound, a radionuclide, and/or a combination thereof. The composition may be prepared prior to administration. The composition may remain in a gelled state during storage, handling, and/or injection. The hydrogel may retain structural integrity under physiological temperature and/or pH. The pre-formed structure may promote reproducible administration. The structure may minimize a need for in situ activation. The structure may enable site-specific delivery using a syringe format.

In some embodiments, the disclosed hydrogel system is developed with consideration of applicable regulatory pathways for localized therapeutic delivery systems comprising radiotherapeutics and/or chemotherapeutics. The formulation, composition, and intended use of the hydrogel may be compatible with United States Food and Drug Administration (FDA) combination product classification. The system may comply with current good manufacturing practices (cGMP). The hydrogel may be regulated as a device-led combination product under Title 21 of the Code of Federal Regulations (CFR), Part 3, and/or related guidance based on its local delivery mechanism and implantable characteristics. The system may align with premarket pathways, e.g., Premarket Notification (510 (k)) and/or De Novo Classification, for localized radiopharmaceutical delivery platforms.

In some embodiments, the hydrogel design reflects parameters consistent with parenteral and/or intratumoral delivery standards. The total volume and rheological profile may be optimized for delivery via standard pre-filled syringes. The composition may comprise generally recognized as safe (GRAS) components and/or United States Pharmacopeia (USP)-grade components suitable for clinical formulation. The components may comprise amylopectin, dimethyl sulfoxide (DMSO), water for injection (WFI), and/or a combination thereof. The total administered radioactivity per injection volume may fall within Investigational New Drug (IND)-suitable dosimetry limits for alpha-emitting radionuclides. The pre-gelled matrix may promote uniform local retention and diffusion control. The matrix may allow integration with therapeutic protocols for Targeted Radionuclide Therapy (TRT), brachytherapy, and/or neoadjuvant radiotherapy.

In some embodiments, the encapsulation and retention profiles demonstrated herein support regulatory engagement through early-stage programs. The programs may comprise the United States Food and Drug Administration (FDA) Initial Targeted Engagement for Regulatory Advice on Combination Products (INTERACT) meeting and/or the Pre-Submission (Pre-Sub) program. The hydrogel device's modular encapsulation platform may allow radionuclide labeling with multiple alpha-emitting radionuclides, beta-emitting radionuclides, and/or a combination thereof. The labeling may support therapeutic and/or theranostic purposes. The platform may provide flexibility under a single delivery system.

In some embodiments, the disclosed results support development of the hydrogel for intratumoral injection in solid tumor indications. The indications may comprise pediatric neuroblastoma, glioma, unresectable malignancies, recurrent malignancies, and/or a combination thereof. The demonstrated encapsulation efficiency and/or radionuclide retention may support further evaluation of the hydrogel system in advanced therapeutic settings. The composition is compatible with surrogate cerium and/or radioactive cerium radionuclides. The encapsulation efficiency (demonstrated herein) and/or radionuclide retention support evaluation of the hydrogel system in advanced therapeutic applications. The composition may be compatible with surrogate cerium and radioactive cerium radionuclides. The system may be suitable for use with actinium-225 (\hat{ }225Ac) and/or other high-linear energy transfer (LET) radionuclides, and/or a combination thereof. The hydrogel system may meet criteria associated with accelerated regulatory review under orphan drug designation and/or breakthrough therapy designation. The criteria may comprise a favorable safety profile, a modular loading workflow, and/or a combination thereof.

In some embodiments, a starch-based hydrogel composition is used as a pre-gelled delivery matrix for encapsulation and localized release of therapeutic radionuclides. The pre-gelled formulation may comprise a 10 wt % amylopectin-dimethyl sulfoxide (DMSO) hydrogel. The formulation may be housed in a syringe system, e.g., 10 uL, for direct intratumoral administration. The hydrogel may enable encapsulation of radionuclides with high efficiency, e.g., greater than 90%. The hydrogel may exhibit sustained release of activity under physiological conditions. The formulation and associated methods may support early preclinical evaluation and/or subsequent Investigational New Drug (IND) submission for targeted radionuclide therapy (TRT) indications. The composition may fall within regulatory classifications associated with combination products. The classification may involve both drug elements and/or device elements based on the therapeutic payload and delivery mechanism. The regulatory pathway may require pre-submission review to determine final designation and applicable approval requirements.

In some embodiments, the present disclosure relates to compositions and delivery systems for localized treatment of solid tumors. Cancer remains one of the leading causes of death worldwide. Many standard-of-care interventions may rely on systemic chemotherapy, radiation therapy, surgical resection, and/or a combination thereof. Systemic chemotherapy may enable drug exposure at metastatic and inaccessible sites. The exposure may cause dose-limiting toxicities and/or adverse effects due to off-target distribution. Radiation therapy may provide precise delivery. Radiation therapy may also damage surrounding healthy tissues, particularly in pediatric tumors and/or surgically inaccessible tumors. These limitations may prompt interest in local, sustained, and/or tumor-selective treatment modalities. The modalities may reduce systemic burden and/or maximize local therapeutic effect.

In some embodiments, injectable hydrogels are explored as carriers for site-specific drug delivery. Hydrogels are soft, water-containing polymer networks. The networks may retain therapeutic payloads and/or release the payloads over time in situ. Localized delivery using injectable hydrogels may reduce systemic exposure and/or prolong tumor-specific drug retention. Several synthetic and natural polymers may be used for hydrogel formation. The polymers may comprise polyethylene glycol, alginate, hyaluronic acid, starch, and/or a combination thereof. Starch-based hydrogels may offer advantages due to biocompatibility, biodegradability, and/or formulation flexibility. The materials may be tailored to incorporate therapeutic agents. The agents may comprise chemotherapeutic compounds, radionuclides, and/or a combination thereof.

In some embodiments, the disclosed hydrogel composition comprises a polysaccharide matrix derived from starch. The polymer component may be selected from amylose, amylopectin, starch blends, and/or a combination thereof. The polymer may be processed to exhibit a controlled molecular weight distribution. The distribution may enable formation of a mechanically stable and injectable gel when combined with suitable solvents and crosslinking agents. The starch-based matrix may be crosslinked using biocompatible acids, e.g., d-glucaric acid. The crosslinking may form a three-dimensional scaffold that promotes sustained retention of hydrophilic agents, lipophilic agents, and/or a combination thereof. The gelation process may occur under ambient or cooled conditions. The process may be compatible with post-processing steps such as sterile filtration and/or syringe loading.

In some embodiments, the hydrogel comprises dimethyl sulfoxide (DMSO) and/or another polar aprotic solvent that is miscible with water and promotes solubilization of poorly water-soluble drugs. DMSO may enhance compatibility of chemotherapeutic agents, e.g., doxorubicin in free base form, with the starch matrix. The formulation may exhibit high encapsulation efficiency for chemotherapeutic payloads and/or radiotherapeutic payloads. The formulation may resist phase separation and/or aggregation under storage and physiological conditions. Upon injection into a tumor site, the hydrogel may form a local depot. The depot may provide prolonged residence and site-specific therapeutic effect.

In some embodiments, the hydrogel is designed for intratumoral injection. The hydrogel may be delivered using a pre-filled syringe. The composition may remain stable in storage for several weeks. The composition may be compatible with image-guided surgical procedures and/or interventional procedures. Upon administration, the hydrogel may form a soft, conformal mass. The mass may remain localized in a tumor region and may resist enzymatic degradation over clinically relevant timeframes. This platform may enable dual-agent delivery. The dual delivery may comprise chemotherapeutic agents, radionuclides, and/or a combination thereof within a single matrix. The radionuclide may emit alpha particles, beta particles, Auger electrons, and/or a combination thereof. The chemotherapeutic component may act via a complementary cytotoxic mechanism. This dual modality may provide synergistic tumor kill and/or reduced systemic toxicity.

In some embodiments, the formulation strategy supports delivery of radionuclides The hydrogel matrix may entrap the radionuclide via steric sequestration and solvent polarity modulation. The matrix may retain the radiotherapeutic dose at an injection site. The composition may emit detectable signals such as fluorescence, Cherenkov radiation, gamma emission, and/or a combination thereof. The signals may enable non-invasive visualization and confirmation of delivery. In some embodiments, the imaging feedback supports intraoperative assessment and/or postoperative monitoring of therapeutic placement. These technical features may allow improved tumor targeting, reduced dose variability, and/or enhanced therapeutic index.

In some embodiments, the platform is designed to meet regulatory and clinical translational goals. The goals may include compatibility with pediatric tumor treatment. The composition may be adapted for use in surgical oncology workflows. The workflows may include intraoperative injection into a tumor cavity and/or post-resection residual disease treatment. The hydrogel may meet quality control criteria for viscosity, clarity, payload stability, and/or syringeability. The formulation may avoid components associated with immunogenicity and/or toxic byproducts. The formulation may be manufactured under aseptic conditions using pharmaceutical-grade precursors.

In some embodiments, the following terminology may be used throughout the present disclosure to aid in interpretation and clarity. The term “hydrogel” refers to a hydrated, crosslinked polymer matrix. The matrix may form a three-dimensional structure that retains aqueous solutions, solvent-based solutions, and/or a combination thereof. The term “depot” refers to a formulation that remains localized at an injection site. The depot may release its contents over time under physiological conditions. The term “encapsulation efficiency” refers to a percentage of a therapeutic agent that remains associated with the matrix upon initial formulation. The term “retention” refers to a percentage of agent that remains within the matrix under defined physiological conditions after a defined time period. The term “radionuclide” refers to an radionuclide that emits ionizing radiation. The radiation may comprise alpha particles, beta particles, Auger electrons, and/or a combination thereof. The radionuclide may be incorporated within the hydrogel matrix for therapeutic and/or diagnostic purposes.

In some embodiments, the hydrogel compositions, formulation techniques, delivery methods, and therapeutic applications disclosed herein provide a novel and clinically translatable platform. The platform may support localized delivery of chemotherapeutic agents, radiotherapeutic agents, and/or a combination thereof to solid tumors. The composition may address limitations associated with physically assembled gels and/or synthetic polymers. The composition may utilize a biocompatible starch-derived matrix. The matrix may enable stable encapsulation, syringe administration, and/or prolonged intratumoral retention. The formulation process may avoid harsh initiators and may support sterile handling. The formulation may be suitable for direct use in surgical workflows and/or interventional oncology workflows.

In some embodiments, the hydrogel matrix is formed from starch-derived polymers. The polymer component may comprise amylose, amylopectin, a starch-derived polysaccharide blend, and/or a combination thereof. The polymer may be obtained from plant-based sources. The polymer may be processed to yield a defined molecular weight distribution suitable for hydrogel formation. The amylopectin fraction may comprise highly branched glucose residues. The branched structure may promote internal crosslinking, matrix cohesion, resistance to passive diffusion, and/or a combination thereof. The structure may enhance retention of therapeutic agents, reduce swelling in aqueous environments, and/or maintain shape, during intratumoral residence. The starch composition may be selected to promote mechanical stiffness, shear-thinning behavior, and/or compatibility with pre-filled injection systems. The use of a plant-derived, pre-formed amylopectin-based matrix may enable tunable gelation without thermal triggers and/or eliminate the need for synthetic PEG or gelatin carriers.

In some embodiments, the hydrogel matrix is crosslinked using a multi-functional crosslinking agent. The crosslinking agent may comprise d-glucaric acid, a polycarboxylic acid, a sugar acid derivative, and/or a combination thereof. D-glucaric acid may provide multiple carboxyl groups, hydroxyl groups, and/or a combination thereof. The functional groups may react with the hydroxyl functionality of starch polymers. The crosslinking reaction may proceed under mildly acidic pH, neutral pH, and/or a combination thereof. The reaction may occur with minimized use of initiators, catalysts, and/or heating. The crosslinking agent may be added to a starch solution under defined pH and/or temperature to promote formation of a covalently entangled network. The resulting hydrogel matrix may comprise a stable three-dimensional structure. The structure may resist shear stress, dilution, and/or enzymatic degradation under physiological conditions. The extent of crosslinking may modulate storage modulus, therapeutic retention, and/or syringe extrusion force. The use of a sugar-derived acid may enable stable gel formation with minimized synthetic PEG linkers, aldehyde-based gelatin systems, and/or thermal crosslinking methods. The crosslinked matrix may improve in vivo persistence, prolong drug release, and/or prevent premature disassembly at the tumor site.

In some embodiments, the solvent is dimethyl sulfoxide (DMSO). The solvent may comprise DMSO, a water-miscible polar aprotic solvent, and/or a combination thereof. The solvent may swell the starch polymer matrix. The solvent may enable solubilization of a hydrophobic therapeutic payload. The solvent may enable loading of a chemotherapeutic compound in its free base form. The solvent may prevent precipitation and/or crystallization of the drug during hydrogel formation. The DMSO content may be at least about 20% v/v, 30% v/v, 40% v/v, and/or 50% v/v. The DMSO content may be at most about 60% v/v, 70% v/v, 80% v/v, and/or 90% v/v. The DMSO content may be of any value between the aforementioned values, e.g., from about 40% v/v to about 60% v/v. The solvent may promote uniform dispersion of doxorubicin, a radionuclide, and/or a combination thereof. The solvent phase may remain stable within the hydrogel matrix during storage, syringe filling, and/or in vivo delivery. The use of DMSO may eliminate the need for micellar encapsulation, emulsifiers, and/or co-solvents during formulation.

In some embodiments, the hydrogel comprises at least one chemotherapeutic agent. The chemotherapeutic agent may comprise doxorubicin in its free base form, a lipophilic analog, and/or a combination thereof. The compound may be solubilized directly in dimethyl sulfoxide. The composition may minimally require, (e.g., not require), conversion to a water-soluble acid salt. The agent may be dispersed during hydrogel formation. The agent may be retained in a non-crystalline state within the matrix. The solvent-swollen polymer network may prevent phase separation, crystallization, and/or degradation of the chemotherapeutic compound. The agent may remain bioavailable during storage, injection, and/or post-administration residence. The composition may enable local intratumoral administration of doxorubicin using a single syringe and/or without requiring systemic exposure.

In some embodiments, the hydrogel comprises a radionuclide as a therapeutic and/or diagnostic agent. In some embodiments, the hydrogel composition comprises a radionuclide selected from alpha-emitting, beta-emitting, gamma-emitting, and/or neutron-activated radionuclides. Suitable alpha-emitting radionuclides include medical radionuclides. The radionuclide may emit alpha particles, beta particles, and/or Auger electrons. The radionuclide may be selected based on its emission energy and/or half-life. The radionuclide may be loaded with or without the use of a chelating ligand. The radionuclide may remain physically entrapped within the crosslinked polymer network. The matrix may retain the radionuclide in a hydrated state and/or under physiological shear. In some embodiments, the composition comprises both a chemotherapeutic compound and a radionuclide. The hydrogel may deliver the agents simultaneously to a tumor site and/or enable temporally synchronized release. The composition may reduce systemic exposure, enable pediatric applications, and/or allow multi-modal local therapy using a single injectable depot.

In some embodiments, the composition is formed by combining a starch-based polymer, a solvent, and/or a therapeutic agent, under ambient or cooled conditions. The components may be mixed to form a hydrogel prior to packaging. Th components may be combined within a pre-filled syringe. The composition may be sterilized by filtration, gamma irradiation, and/or aseptic manufacturing. The hydrogel may retain single-phase uniformity. The hydrogel may remain visually homogeneous during cold storage, mechanical agitation, and/or clinical handling. The composition may be filled into a syringe in its final gelled state. The composition may minimally require, (e.g., not require), thermal activation, mixing, and/or reconstitution prior to use. The hydrogel may be free of visible aggregates, fibrous residues, and/or entrapped air bubbles. The hydrogel may be injectable through a needle having an inner diameter of at least about 25 gauge (25G), 27G, 29G, and/or 30G. The hydrogel may maintain consistent extrusion behavior and/or therapeutic uniformity during injection. The filled syringe may remain stable for at least about two weeks, three weeks, and/or four weeks. The filled syringe may remain stable for at least about two weeks, three weeks, and/or four weeks under ambient temperature.

In some embodiments, the composition is a hydrogel that is stable during storage. The hydrogel may remain stable for at least about 2 weeks, 3 weeks, 4 weeks, and/or 6 weeks. The storage temperature may be at least about 2 degrees Celsius (C), 4° C., 6° C., and/or 8° C. The storage temperature may be at most about 10° C., 12° C., 15° C., and/or 20° C. The storage temperature may be of any value between the aforementioned values, e.g., from about 4° C. to about 8° C. The hydrogel may retain viscosity, gelation state, and/or therapeutic agent concentration, with minimal (e.g., without) undergoing visible phase separation, crystallization, and/or degradation. The therapeutic payload may remain physically and chemically stable under storage conditions. The pre-filled syringe may be inspected prior to use and may meet acceptance criteria for color, opacity, clarity, and/or absence of bubbles.

In some embodiments, the hydrogel composition comprises a polymer matrix, a solvent, and/or a therapeutic payload. The matrix may comprise crosslinked starch-derived polymers. The matrix may form a mechanically cohesive scaffold for solvent and/or agent retention. The solvent may comprise a polar aprotic liquid such as dimethyl sulfoxide. The solvent may remain distributed within the hydrogel under ambient and/or refrigerated conditions. The therapeutic payload may comprise a chemotherapeutic compound, a radionuclide, and/or a combination thereof. The therapeutic agents may be co-localized within the matrix. The agents may remain stable without phase separation, degradation, and/or crystallization. The composition may be formed prior to injection. The composition may require minimal (e.g., not require) in situ gelation, chelator conjugation, and/or polymerization triggers. The formulation may exclude synthetic carriers such as polyethylene glycol (PEG) and/or gelatin. The formulation may remain compatible with single-dose, pre-filled injection formats.

In some embodiments, a hydrogel composition comprises a polymeric matrix, a solvent phase, and/or at least one dispersed agent within the matrix. The matrix may comprise a starch-derived polymer such as amylopectin. The matrix may define a continuous network structure. The solvent phase may comprise dimethyl sulfoxide and/or a water-miscible carrier. The carrier may swell the matrix and/or promotes therapeutic dispersion. The dispersed agents may comprise chemotherapeutics, radionuclides, diagnostic radionuclides, and/or crosslinkers, that modify the hydrogel's behavior. The behavior modification may include chemical and/or physical modification. The components may be combined to form a stable, single-phase hydrogel prior to injection. The formulation may be stored, handled, and/or delivered using a pre-filled syringe. The matrix, solvent, and/or payload components may interact to influence various factors. The factors may comprise mechanical stiffness, injectability, drug retention, and/or tissue residency, of the hydrogel after administration. In some embodiments, the interaction between hydrogel composition and therapeutic payload may determine clinical applicability. The matrix, solvent, and agent formulation may collectively influence diffusion, degradation, and/or tissue residence. These factors may contribute to therapeutic performance in localized cancer treatment applications.

In some embodiments, the hydrogel composition promotes therapeutic benefit through local retention of alpha-emitters. The alpha-emitters may activate antitumor immune responses. The retained radionuclide may trigger immunogenic cell death and/or tumor-associated antigen release. The formulation may reduce systemic immune suppression. The formulation may promote durable tumor control through innate and/or adaptive immune mechanisms.

In some embodiments, the starch-based hydrogel comprises a formulation platform developed for intratumoral delivery of chemotherapeutic agents. The composition may be designated as Amygel. The composition may comprise a 10 wt % amylopectin matrix solvated in dimethyl sulfoxide. The composition may enable sustained release of doxorubicin and/or be adapted to encapsulate therapeutic radionuclides without modifying the underlying gel chemistry.

In some embodiments, the composition supports integration with targeted alpha therapies. The therapies may comprise diffusing alpha-emitters radiation therapy (DaRT). The DaRT approach may comprise localized administration of alpha-emitting radionuclides, e.g., 224Ra. The radionuclides may exhibit short half-lives and/or favorable diffusion profiles. The composition may enable localized placement of the radionuclides within tumor tissue. The composition may enhance tumor response and/or reduce acute toxicity, delayed toxicity, and/or a combination thereof. In some embodiments, formulation performance and agent retention are characterized using analytical techniques. Evaluation methods may comprise spectrophotometric analysis, structural assessment, and/or radioactivity quantification. These methods may confirm encapsulation behavior, matrix compatibility, and/or loading reproducibility.

In some embodiments, the encapsulation efficiency, retention behavior, and formulation compatibility demonstrated with cerium radionuclides supports extension to alpha-emitting radionuclides such as actinium-225 (\hat{ }225Ac). Actinium-225 may provide high-linear energy transfer (LET) emission profiles with therapeutic relevance for localized alpha therapy. The hydrogel matrix may retain actinium-225 without requiring chelation and/or covalent modification. The matrix may enable site-specific delivery, reduced systemic redistribution, and/or sustained intratumoral residence of −225Ac. These findings may support evaluation of the hydrogel platform for alpha particle-based treatments, including those targeting radioresistant and/or surgically inaccessible tumor types.

In some embodiments, pre-gelled starch-based hydrogel samples are assessed for encapsulation of non-radioactive cerium ions as a model compound for radionuclides. The hydrogel matrix may enable uptake under static and/or dynamic mixing conditions. Retention of cerium ions may be evaluated using UV-Vis spectroscopy and/or surrogate indicators, e.g., methylene blue. The assessment may confirm compatibility with aqueous salt solutions. The assessment may establish baseline calibration for subsequent radiolabeling studies. The studies (described herein) demonstrate compatibility of the hydrogel composition with aqueous cerium solutions, agent loading conditions, and/or physicochemical parameters relevant to therapeutic formulation.

In some embodiments, encapsulation efficiency of radionuclides is enhanced under dynamic mixing conditions. The reaction may be performed at approximately 40° C. using a rotation speed of approximately 15000 rpm for a duration of approximately 30 minutes. A 5 μL aliquot of a 100 mM radionuclide solution may be introduced into a dimethyl sulfoxide-based hydrogel containing approximately 50 mg of pre-formed polymer. The resulting hydrogel formulation may exhibit improved uptake, reproducibility, and loading uniformity relative to static protocols.

In some embodiments, the hydrogel is formed using a starch-based polymer such as amylopectin. The polymer may be solvated in dimethyl sulfoxide and microwave-treated to yield a gel matrix with defined mesh size and porosity. The resulting hydrogel may be stored at approximately 4° C. and used directly for metal ion and/or radionuclide encapsulation without further processing. The formation conditions may support stable retention of hydrophilic payloads and/or may enable syringe administration.

In some embodiments, surrogate cation loading solutions are prepared using cerium chloride dissolved in water, methanol, dimethyl sulfoxide, and/or a combination thereof. The cerium chloride solution may be introduced into the hydrogel by pipetting. The solution may be incorporated via vortexing and/or mechanical agitation. Encapsulation may be confirmed using UV-Vis absorbance measurements validated against standard calibration curves (described herein). The resulting formulations may define concentration, loading volume, and/or agitation parameters compatible with high-efficiency encapsulation and uniform distribution. In some embodiments, surrogate loading outcomes may inform formulation design for radioactive analogs. The observed retention, structural integrity, and solvent compatibility may support the suitability of starch-based hydrogels for therapeutic radionuclide incorporation under clinically relevant conditions.

In some embodiments, a hydrogel formulation comprising Amygel, dimethyl sulfoxide (DMSO), and doxorubicin (DOX) in a free base form is used for local chemotherapy delivery. The DMSO solvent may enhance solubilization of the lipophilic DOX molecule. The solvent may allow a polymer matrix to retain high concentrations of drug. The free base form of anthracyclines may penetrate tumor tissue up to 10 mm following a single 10 uL intratumoral injection. The formulation disclosed herein may demonstrate a multifold improvement in tumor penetration relative to conventional delivery systems.

In some embodiments, a single image-guided intratumoral injection of a DOX-Amygel formulation produces sustained distribution of drug throughout a tumor mass.

In some embodiments, ultrasound is used to guide intratumoral injection of Amygel formulations and/or to monitor tumor response. The hydrogel may be visible in situ throughout an imaging period. Tumor response may include visible necrosis regions. The regions may be identified by ultrasound and confirmed by histological examination. In anthracycline-loaded Amygel formulations, ex vivo fluorescence imaging may demonstrate diffusion of drug up to 10 mm from an injection site. The treated zones may correspond to histologically confirmed necrosis regions.

The aforementioned studies, evaluations, and/or analyses provide quantitative support for encapsulation efficiency, sustained release behavior, and/or physicochemical stability of radionuclide-loaded hydrogels.

In some embodiments, the hydrogel matrix comprises a three-dimensional network formed by crosslinked starch-derived polymers. The matrix may comprise amylopectin chains joined through a sugar-based crosslinking agent such as d-glucaric acid. The crosslinked network may promote mechanical stiffness, resist dissolution under physiological conditions, and/or retain internal pore structure during storage. The matrix may physically entrap a therapeutic agent. The matrix may reduce diffusion, leakage, and/or systemic release following administration. The internal structure of the matrix may enable uniform distribution of agents without chelator conjugation and/or synthetic encapsulation. The matrix may remain hydrated. The matrix may maintain therapeutic loading under temperature cycling, shear, and/or injection through narrow-gauge needles.

In some embodiments, the composition is delivered by local intratumoral injection to a solid tumor. The hydrogel may be injected directly into a tumor mass. The tumor may conform to anatomical irregularities within a lesion without diffusing into surrounding tissue compartments. The composition may form a depot that physically retains therapeutic agents. The composition may maintain uniform local concentrations within a tumor volume. The matrix may remain stable under physiological shear, pH, and/or enzymatic activity. The localized administration may allow delivery of high-dose chemotherapeutic and/or radiotherapeutic agents without systemic dose limitations. The composition may be used in tumors that are surgically inaccessible and/or adjacent to critical structures. The composition may provide spatially restricted therapy with minimal systemic burden. The hydrogel may be injected intraoperatively and/or under image guidance. The hydrogel may remain at a site of injection for at least about 3 days, 7 days, 10 days, and/or 14 days. The tumor may comprise a soft-tissue lesion, e.g., brain, and/or a locally recurrent neoplasm, e.g., medulloblastoma.

In some embodiments, the composition exhibits low systemic bioavailability following local administration. The hydrogel may retain therapeutic agents within a tumor mass. He hydrogel may reduce leakage into circulation through passive matrix entrapment. The composition may comprise a crosslinked network that hinders diffusion of low molecular weight compounds and/or ionic species into a bloodstream. In some embodiments, systemic radioactivity is below detectable levels at 24 hours post-injection. The systemic signal may be measured by gamma counting, plasma scintillation, and/or radiometric analysis of biological fluids, e.g., blood. The radionuclide may be retained without covalent conjugation, chelator encapsulation, and/or chemical modification. The retention mechanism may depend at least in part on steric hindrance, solvent affinity, and/or physical entrapment within a gel structure. The composition may support therapeutic use of radionuclides with systemic toxicity profiles. The composition may reduce off-target exposure to radiosensitive organs.

In some embodiments, the composition is administered as a single intratumoral injection. The composition may provide a therapeutic effect that persists for multiple days. The hydrogel may deliver a chemotherapeutic agent, a radionuclide, and/or a combination thereof, in a spatially confined depot. The depot may suppress tumor growth while minimizing (e.g. substantially without) the need for systemic administration, continuous infusion, and/or repeated dosing. In vivo studies in solid tumor models demonstrate extended survival following a single dose of the hydrogel composition. The composition may prolong median survival and/or increase long-term survival fractions in murine subjects bearing orthotopic and/or flank tumors. The composition may comprise doxorubicin, actinium-225, and/or cerium-144 retained within the matrix over a period of at least about 3 days, 7 days, 10 days, and/or 14 days. The survival outcome may depend at least in part on sustained intratumoral retention and/or the simultaneous presence of cytotoxic and/or radiotherapeutic agents.

In some embodiments, the therapeutic effect arises from a localized depot that maintains agent concentration at the tumor site without detectable systemic redistribution. The hydrogel matrix may form a stable structure that resists dissolution, fragmentation, and/or lymphatic clearance. The composition may be formulated without stabilizing excipients, chelators, emulsifiers, and/or synthetic carriers. The composition may retain biological activity while minimizing (e.g., substantially without) in situ polymerization and/or drug conjugation. The therapeutic agents may remain active within the depot. The therapeutic agents may continue acting on tumor tissue during the full duration of their retention. The depot may suppress local recurrence and/or delay progression of residual lesions, following surgical resection and/or biopsy. The efficacy of the hydrogel may exceed that of equivalent free drug and/or unformulated radionuclide. The efficacy of the hydrogel may reflect synergistic or additive interactions between agents retained within a spatially restricted domain.

In some embodiments, the therapeutic agent remains localized at the tumor site for an extended duration following injection. The composition may retain fluorescent agents, chemotherapeutic compounds, and/or radiolabeled surrogates within the hydrogel matrix. Fluorescence imaging of injected tumors indicates that signal intensity remains detectable for at least about 3 days, 5 days, and/or 7 days after administration. The fluorescent signal may remain spatially confined to the tumor mass. The fluorescent signal may not diffuse into surrounding soft tissue compartments. The intensity decay over time may reflect agent clearance through biological degradation and/or matrix erosion, rather than systemic leakage. The retained signal may be visible under in vivo imaging modalities such as fluorescence stereomicroscopy and/or ex vivo confocal microscopy. The signal profile may correlate with sustained therapeutic activity. The signal profile may enable tracking of agent persistence while minimizing (e.g., without) the use of external imaging tracers or synthetic fluorophores.

In some embodiments, the observed fluorescence retention results at least in part from a combination of physical entrapment, solvent affinity, and/or matrix stabilization. The hydrogel may retain hydrophilic and/or amphiphilic agents without requiring covalent tethering, chemical conjugation, and/or metal chelation. The polymeric network may provide spatial confinement without additional nanoparticle encapsulation and/or depot-forming components. In some embodiments, fluorescence remains localized within the gel, with no detectable systemic distribution. The agent may remain immobilized within the hydrogel without relying on cellular uptake, receptor binding, and/or active localization. This passive retention may provide a therapeutically relevant confinement period for solid tumors in anatomically restricted regions.

In some embodiments, the composition comprises a medical radionuclide that remains physically localized at the injection site after administration.

In an example, post-injection imaging and radiometric analysis, confirm that radioactivity is confined to the tumor region for a period of at least about 1 day, 3 days, 5 days, and/or 7 days. The composition may exhibit minimal systemic redistribution, with no detectable signal in blood plasma, liver, kidney, and/or bone marrow samples. Retention may arise from steric exclusion within the crosslinked matrix, physical entrapment within the hydrated gel, and/or charge interactions between the radionuclide species and/or the gel environment. Radioactivity may be measured using gamma scintigraphy, radiometric sampling, and/or liquid scintillation analysis (e.g., whole blood). The composition may be injected intratumorally. The composition may not require intravenous administration, circulation, or vascular targeting to reach the lesion. In some embodiments, the retained signal corresponds to emission intensity measured in situ using small-animal imaging platforms and/or intraoperative detectors.

In some embodiments, the observed radionuclide retention reflects a combination of physical, chemical, and structural features. The hydrogel may localize radionuclides with or without the use of synthetic chelators, nanoparticle encapsulation, and/or active targeting moieties. The composition may differ from conventional formulations that rely on carrier particles, liposomes, and/or antibody conjugates, to retain radionuclides at disease sites. The use of a starch-based hydrogel comprising dimethyl sulfoxide may contribute to radionuclide compatibility by modulating solvation behavior and/or diffusivity. The starch-based hydrogel may offer advantages in biocompatibility, degradation rate, and/or tunability relative to synthetic matrices. The composition may be pre-formed prior to administration. The composition may not depend on temperature- or pH-triggered gelation in vivo. The hydrogel structure may differ from prior art hydrogels that require in situ formation to achieve retention. In some embodiments, the crosslinked network stabilizes the payload without requiring polymerization within the body and/or exposure to initiating conditions (e.g., UV, enzymatic catalysis).

In some embodiments, the hydrogel composition demonstrates superior retention and therapeutic efficacy compared to unformulated controls. In vivo studies indicate that free chemotherapeutic agents and/or unencapsulated radionuclides administered intratumorally may exhibit rapid clearance from the injection site. The chemotherapeutic agents and/or unencapsulated radionuclides may exhibit rapid (e.g., within 24 hours) redistribution to systemic compartments. Fluorescence and/or radiometric imaging reveal that signal intensity associated with free agent formulations declines substantially within the first 24 hours. The signal intensity may become undetectable after 48 hours. In contrast, the hydrogel formulation retains measurable signal for at least about 3 days, 5 days, and/or 7 days under similar conditions. The free agent may diffuse beyond the tumor margin and may accumulate in off-target tissues (e.g., liver, kidney, bone marrow), leading to increased systemic exposure. The observed contrast in signal persistence and/or spatial confinement supports the conclusion that the hydrogel matrix functions as retention scaffold distinct from prior art delivery vehicles.

In some embodiments, the composition retains its depot behavior and retention profile, across multiple experimental replicates and tumor models. The hydrogel may form a spatially localized structure in tumors exhibiting varying degrees of vascularization, necrosis, stiffness, and/or extracellular matrix density. The composition may perform consistently in both orthotopic and/or subcutaneous tumor settings. The composition may maintain therapeutic payload without requiring tumor-specific adaptation. Batches prepared using the same formulation parameters may yield comparable viscosity, gelation behavior, and/or retention performance. In some embodiments, the composition may retain agents in tumor tissue despite local enzymatic degradation, acidic microenvironments, and/or immune cell infiltration. The structural stability and/or biological persistence of the hydrogel may support broader applicability across patient-derived xenografts and/or spontaneous tumor models.

In some embodiments, the hydrogel composition comprises a chemotherapeutic agent and a radionuclide retained within the same matrix volume. The two agents may remain co-localized within the hydrogel without competitive diffusion, chemical interaction, and/or premature release. The composition may provide temporally synchronized delivery of alpha, beta, and/or small-molecule cytotoxic activity at the tumor site. The retention behavior may result from polymer mesh entrapment, solvent-mediated solubility control, and/or interaction with the polymer-solvent interface. In vivo studies suggest that both agents remain at the tumor site for at least about 3 days, 5 days, and/or 7 days with overlapping therapeutic action. The dual-agent configuration may provide additive and/or synergistic effects. The effects may be due to combining DNA damage mechanisms and/or promoting tumor cell clearance beyond what is achievable with either agent alone. The simultaneous retention of dissimilar therapeutic modalities may enable precision scheduling, dose optimization, and/or immune priming without systemic co-exposure.

In some embodiments, the composition differs structurally and functionally from conventional delivery systems. The difference may be based on synthetic polymers, liposomes, and/or particulate carriers. Unlike liposomal formulations, the hydrogel disclosed herein may not require phospholipid membranes, remote loading gradients, and/or cholesterol stabilization. Unlike poly (lactic-co-glycolic acid) (PLGA) microspheres, the matrix disclosed herein may not rely on degradation-driven payload release and/or solvent-triggered phase inversion. The composition may exclude nanoparticle encapsulation, antibody conjugation, and/or engineered targeting domains. In contrast to intravenous agents, the hydrogel may act locally upon administration. The hydrogel may not depend on circulation time and/or vascular permeability to reach the tumor. The matrix may deliver multiple therapeutic agents in a physically stabilized network without requiring in situ gelation and/or carrier additives. These features may distinguish the starch-based composition disclosed herein as a structurally novel system with clinically relevant advantages in site-specific delivery and/or formulation simplicity.

In some embodiments, the composition is suitable for use in pediatric patients with solid tumors. The hydrogel may deliver therapeutic agents directly to the tumor site. The hydrogel may reduce systemic toxicity by avoiding intravenous exposure and/or off-target accumulation. The formulation may be administered locally at the time of biopsy, resection, and/or stereotactic access. The formulation may remain at the tumor site as a depot. In pediatric subjects, the hydrogel may minimize drug exposure to developing organs, hematopoietic tissues, and/or endocrine systems. The depot may retain cytotoxic and/or radiotherapeutic agents within the tumor. The agent retention may not require repeat injections, catheter implantation, and/or systemic maintenance therapy. In some embodiments, the composition may be applied in children with high-risk and/or recurrent tumors. The composition may be applied to patients where systemic chemotherapy has failed and where localized retention is clinically beneficial. The composition may support site-specific treatment while reducing cumulative toxicity burden in vulnerable pediatric populations.

In some embodiments, the composition is applicable to pediatric brain tumors including medulloblastoma, ependymoma, and/or other central nervous system (CNS) neoplasms. The hydrogel may be delivered to tumor tissue located within and/or proximal to the brainstem, cerebellum, ventricles, and/or other intracranial structures. The composition may conform to post-resection cavities and/or residual tumor margins. The composition may remain confined to the anatomical region without diffusing into normal parenchyma and/or cerebrospinal fluid. The use of a pre-formed, shear-thinning hydrogel may enable image-guided and/or intraoperative placement with controlled injection volume. In some embodiments, the hydrogel is compatible with tissues that have undergone prior radiation and/or chemotherapy. The hydrogel may be compatible with tissues that may not require modification of the surrounding blood-brain barrier. The local retention of therapeutic agents may permit treatment of surgically inaccessible and/or radioresistant tumor sites while minimizing off-target neurotoxicity. The local retention of therapeutic agents may preserve functional tissue near eloquent cortical and/or brainstem regions.

In some embodiments, the hydrogel depot retains its physical location and/or structural integrity, when placed near critical anatomical structures. The composition may be injected in proximity to nerves, vasculature, ventricles, and/or functional brain regions. The composition may be injected without exhibiting uncontrolled diffusion, fragmentation, and/or liquefaction. The hydrogel may remain conformal to complex tissue contours. The hydrogel may maintain depot geometry under mild compression, perfusion, and/or tissue motion. In some embodiments, the gel does not migrate into cerebrospinal fluid, vascular compartments, and/or adjacent soft tissue regions. The hydrogel may be retained in the parenchymal and/or subpial space. The crosslinked matrix may limit convection- and diffusion-driven escape of therapeutic agents. The composition may preserve site-specific action while minimizing risk to adjacent tissue. The depot may provide a therapeutic presence along resection margins, within biopsy tracts, and/or near eloquent structures. The depot may provide the therapeutic presence with minimally (e.g., without) requiring mechanical fixation and/or external barriers. In some embodiments, the hydrogel remains stable within post-operative environments. The post-operative environments may be for example, edema, local hemorrhage, and/or cerebrospinal fluid fluctuations. The hydrogel may not require fixation, barrier placement, and/or tissue adhesives in the post-operative environments.

In some embodiments, the hydrogel composition comprises components selected for regulatory acceptability in pediatric applications. The matrix may be free of synthetic polymers, surfactants, stabilizers, and/or excipients, that pose toxicity risks in children. The starch-based polymer may be derived from plant sources that are generally recognized as safe (GRAS). The plant sources may degrade into biocompatible glucose oligomers under physiological conditions. The solvent may comprise dimethyl sulfoxide (DMSO), which is used in approved pediatric drug products at controlled concentrations. In some embodiments, the composition does not require preservatives, crosslinking catalysts, and/or synthetic gelation triggers. The composition may not require any additive that may elicit local and/or systemic adverse effects. The hydrogel may be prepared using aseptic techniques. The hydrogel may be prepared using sterilized post-formation without altering physical properties and/or therapeutic activity of the formulation. The formulation may comply with excipient concentration limits set forth in pediatric regulatory guidance. The formulation may be suitable for intratumoral use in neonates, infants, children, and/or adolescents.

In some embodiments, the hydrogel composition differs structurally and clinically from conventional systemic therapies used for pediatric solid tumors. The formulation may be delivered intratumorally without intravenous infusion, vascular access devices, or systemic circulation. The hydrogel may retain therapeutic payloads within the tumor and reduce exposure to healthy organs. The composition may avoid peak plasma concentrations and dose-limiting systemic toxicities associated with standard agents such as doxorubicin, cisplatin, and/or etoposide. The absence of systemic distribution may reduce the need for hematologic monitoring, central line placement, and/or inpatient administration. In some embodiments, the hydrogel supports a non-systemic treatment strategy that delivers therapeutic effects through local exposure and depot formation, representing a clinically distinct alternative to established protocols for pediatric oncology.

In some embodiments, the hydrogel composition comprises a radionuclide selected to provide localized radiotherapeutic effect at the tumor site. The radionuclide may be selected from alpha-emitting radionuclides, beta-emitting radionuclides, and/or Auger electron-emitting radionuclides. It should be appreciated that any suitable medical radionuclides and/or a combination thereof may be used.

The selected radionuclide may be dispersed within the hydrogel matrix as a free ion, salt, oxide, and/or nanoparticulate form. The selected radionuclide may or may not require chemical chelation and/or encapsulation. The physical retention of the radionuclide within the gel network may permit sustained emission of cytotoxic radiation within the tumor volume. In some embodiments, the radionuclide is selected to achieve a desired tissue penetration profile, emission energy, and/or half-life, suitable for the targeted tumor type. Alpha-emitting radionuclides may provide localized double-stranded DNA damage with minimal lateral spread, while beta emitters may achieve a broader therapeutic radius. The composition may be tailored by radionuclide selection to address tumor geometry, radiosensitivity, and/or proximity to critical tissues.

In some embodiments, the hydrogel composition retains radionuclides through physical entrapment within the matrix, without the use of chelators, encapsulation vesicles, or covalent linkers. The radionuclide may be embedded within the crosslinked starch polymer network and stabilized by electrostatic interaction, hydration shell confinement, and/or steric restriction.

In some embodiments, the hydrogel composition comprises both one or more chemotherapeutic agents and one or more radionuclide co-localized within the same polymer matrix. The composition may comprise a combination of one or more chemotherapeutic agents and one or more radionuclides co-localized within a single polymer matrix. The chemotherapeutic agents may comprise doxorubicin in its free base form and/or other compounds that are substantially insoluble in water and soluble in dimethyl sulfoxide (DMSO). The radionuclides may comprise one or more alpha emitters, e.g., actinium-225, and/or beta emitters, e.g., cerium-144. The therapeutic agents may be suspended and/or solubilized in a shared solvent phase and physically retained within the crosslinked starch matrix without requiring chemical conjugation. In some embodiments, the co-encapsulation of multiple agents enables synergistic cytotoxic effects via complementary mechanisms of action, including DNA intercalation, oxidative damage, and/or high-linear energy transfer (LET)-induced strand breaks. The agents may be released concurrently and/or with staggered kinetics depending at least in part on their interaction with the hydrogel matrix and solvent phase. The composition may support coordinated delivery of multimodal therapy to a localized tumor site while minimizing systemic distribution of each agent. The combination may be applicable to tumors exhibiting resistance to single-agent chemotherapy and/or requiring combinatorial cytotoxic approaches.

In one embodiment, the hydrogel comprises doxorubicin in its free base form at a concentration of at least about 0.1 mg/mL. The doxorubicin concentration is in the range of from about 0.1 mg/mL to about 10 mg/mL. The composition comprises dimethyl sulfoxide as the solvent at a concentration of at least about 20% v/v.

In some embodiments, the hydrogel composition may be adapted to enable boron neutron capture therapy (BNCT) through localized delivery of a boron-containing therapeutic agent. The agent may comprise a boron-rich compound selected for its ability to accumulate within tumor tissue and undergo high-energy alpha emission upon neutron activation. Suitable boron-containing species may comprise carboranes, boronic acids, boronated phenylalanine analogs, and/or a combination thereof. The boron agent may be physically retained within the hydrogel matrix and co-delivered with or without additional therapeutic agents. The pre-formed hydrogel may be injected intratumorally or along surgical margins prior to neutron exposure using a reactor or accelerator-based neutron source. In some embodiments, the gel acts as a localized depot to concentrate boron atoms at the tumor site while minimizing uptake by surrounding healthy tissues. In some embodiments, the composition comprises a boron-containing hydrogel formulation suitable for boron neutron capture therapy (BNCT). The formulation may be delivered intratumorally and may retain the boron-based therapeutic agent under physiologic conditions. BNCT activation may involve external neutron irradiation at a clinical facility equipped with appropriate neutron-generating infrastructure. Although in vivo BNCT activation data are not yet available, the composition may support site-specific BNCT treatment strategies for pediatric and/or adult solid tumors.

In some embodiments, the hydrogel composition comprises a boron-containing therapeutic agent for use in boron neutron capture therapy (BNCT). The agent may comprise boronophenylalanine (BPA), sodium borocaptate (BSH), and/or a combination thereof. The boron-containing agent may be loaded into a pre-gelled starch-based hydrogel using static or dynamic encapsulation methods. The agent may be retained within the matrix through physical entrapment, solvent affinity, and/or ionic interaction. The composition may be injected directly into a tumor mass to localize the boron payload prior to neutron activation. In some embodiments, the boron-containing hydrogel composition may be evaluated using surrogate encapsulation and retention studies to confirm compatibility with site-specific loading and sustained release. Static and/or dynamic protocols may be applied to assess loading efficiency, matrix stability, and agent retention over time. The matrix may maintain gel structure and steric integrity under encapsulation conditions. The hydrogel may prevent premature diffusion of boron agents into surrounding tissue and may provide a defined depot for neutron irradiation.

In some embodiments, BNCT treatment is performed by administering the boron-loaded hydrogel to a target lesion followed by exposure to an external neutron source. The neutron flux may be generated using a hospital-based accelerator or a research-grade neutron generator. Upon neutron absorption, the boron-10 radionuclide may undergo nuclear fission, generating high-LET alpha particles and lithium nuclei that cause localized cytotoxicity within the tumor. The starch-based hydrogel may retain the boron agent in spatial proximity to malignant cells and may reduce off-target deposition in healthy tissues. These features may support application of BNCT in pediatric tumors, surgically inaccessible lesions, and/or locally recurrent cancers.

In some embodiments, the hydrogel composition is stored and administered using a pre-filled syringe suitable for localized intratumoral delivery. The syringe may comprise a barrel volume of at least about 0.5 mL, 1.0 mL, and/or 2.0 mL, and may be at most about 5.0 mL, 10.0 mL, and/or 20.0 mL. The volume may be of any value between the aforementioned values, e.g., from about 1.0 mL to about 5.0 mL. The syringe may be fabricated from polypropylene, glass, and/or another radiation-compatible polymer. The syringe may be stored under refrigerated conditions in a sealed primary container. The hydrogel may be stable within the syringe for at least about 2 weeks, 3 weeks, and/or 4 weeks without phase separation, degradation, and/or therapeutic loss. In some embodiments, the syringe is configured for single use. The syringe may be compatible with image-guided delivery methods including CT-, MRI-, and/or ultrasound-assisted injection. The needle gauge may be at least about 25G, 27G, and/or 30G. The needle gauge may support smooth extrusion of the hydrogel without clogging or deformation. The delivery format may enable outpatient administration and/or minimize the need for compounding or specialized preparation equipment.

In some embodiments, the hydrogel composition comprises a radionuclide selected for compatibility with standard storage, shielding, and handling protocols. The composition may be stored in radiation-shielded syringes and/or vials. The composition may exhibit minimal external radiation exposure due to self-attenuation by the hydrogel matrix. The physical entrapment of the radionuclide within the gel may limit the emission of secondary radiation. The entrapment may reduce the risk of surface contamination and/or aerosolization, during preparation and administration. In some embodiments, the composition supports safe handling in outpatient, surgical, and interventional radiology settings without requiring containment cabinets or advanced shielding infrastructure. The radiation profile may be characterized by a short path length and low off-target emission, permitting storage, transport, and disposal using existing protocols for sealed or encapsulated medical radionuclides.

In some embodiments, a method of treating a solid tumor comprises administering a hydrogel composition directly into the tumor or a resection cavity. The hydrogel may comprise a starch-derived polymer matrix, a polar aprotic solvent, and at least one therapeutic agent selected from a chemotherapeutic compound, a radionuclide, and/or a combination thereof. The composition may be prepared as a pre-formed injectable gel and loaded into a syringe for direct application. The method may comprise injecting the composition into a pediatric or adult tumor using image-guided delivery techniques, and/or during surgical intervention. The hydrogel may form a localized depot that retains therapeutic agents at the injection site and limits systemic exposure. In some embodiments, the hydrogel remains structurally stable under physiological conditions and continues to deliver therapeutic effect over a period of hours, days, and/or weeks following injection. The method may be applied to treatment of medulloblastoma, ependymoma, sarcoma, or other locally accessible tumor types.

In some embodiments, the disclosed hydrogel compositions exhibit dose-responsive survival effects when administered intratumorally in preclinical animal models. Single-dose survival profiles may vary based on drug loading and initial tumor burden, while multi-dose regimens may enhance therapeutic duration and cumulative efficacy. The survival responses may depend at least in part on localized drug retention, hydrogel residence time, and/or tumor-specific pharmacodynamics. The hydrogel formulation may provide sustained therapeutic exposure at the tumor site while minimizing systemic toxicity. Kaplan-Meier curves may be used to evaluate therapeutic selectivity, delayed recurrence, and/or time-to-event endpoints relevant to clinical translation. These data demonstrate the potential of Amygel formulations to support precision-dosing strategies and long-term tumor suppression through localized, injectable chemotherapy delivery.

In some embodiments, ex vivo imaging of a tumor tissue section demonstrates penetration of a chemotherapeutic agent following local administration of a pre-formed hydrogel composition. The hydrogel may comprise a starch-based polymer matrix and/or dimethyl sulfoxide, and may retain and distribute a therapeutic payload, e.g., doxorubicin, throughout tumor tissue following injection. The therapeutic agent may exhibit diffusion from the hydrogel depot into surrounding tumor regions and/or distribute into necrotic or vascularized compartments. Penetration distance may exceed at least about 2 mm, 4 mm, 6 mm, and/or 8 mm. Penetration distance may be at most about 10 mm, 12 mm, 14 mm, and/or 16 mm. Penetration distance may be of any value between the aforementioned values, e.g., from about 6 mm to about 10 mm. The image supports localized retention with effective volumetric dispersion of the drug, thereby illustrating enhanced intratumoral coverage without systemic leakage. The result provides support for the novelty of localized depot-based chemotherapy delivery enabled by the disclosed hydrogel platform.

In an example, pharmacokinetic (PK) evaluation of a doxorubicin-loaded starch-based hydrogel is performed using a murine xenograft model under controlled intratumoral injection conditions. The hydrogel composition comprises a 10 wt % amylopectin matrix formulated in dimethyl sulfoxide (DMSO), and doxorubicin is loaded by passive swelling. Intratumoral administration is conducted in immunodeficient nude mice bearing established subcutaneous tumors. Post-injection plasma samples are collected at multiple time points ranging from 0.125 hours to 24 hours. In some embodiments, bioanalytical quantification is performed using a liquid chromatography-tandem mass spectrometry (LC-MS/MS) assay. The assay employs a Phenomenex® Kinetex® C18 column (2.6 μm, 50 mm×2.1 mm) operated with a linear gradient from 10% to 100% acetonitrile containing 0.1% formic acid. Doxorubicin is detected using positive ion electrospray ionization in multiple reaction monitoring (MRM) mode with a 544.2+397.0 transition, and daunorubicin is used as an internal standard with a 528.2+321.1 transition. The lower limit of quantification (LLOQ) is 1 ng/ml. Assay precision is validated across quality control levels with coefficients of variation below 8.9%. Calibration curves are linear from 1 ng/ml to 2500 ng/ml, with r² values greater than 0.997. In some embodiments, PK parameters are calculated using noncompartmental analysis in Phoenix® WinNonlin® software. The concentration-time profile reveals a tri-phasic disposition with a maximum plasma concentration (Cₘₐₓ) occurring at 0.125 hours post-injection. The elimination half-life (t1/2) is calculated to be approximately 12.2 hours based on the log-linear terminal slope. The apparent volume of distribution (Vz/F) is estimated at 1230 L/kg, and the apparent clearance (CL/F) is calculated at 70 L/h/kg. Percent coefficient of variation for Cₘₐₓ ranges from approximately 9% to 56% across biological replicates.

In an example, systemic bioavailability of doxorubicin is estimated by comparison to published intravenous data under equivalent murine conditions. The relative bioavailability (F) of the intratumoral hydrogel formulation is approximately 7%, indicating effective local retention of the active agent and minimal systemic leakage. The low F value aligns with depot-based release and gradual outward diffusion from the hydrogel matrix. In some embodiments, this pharmacokinetic dataset supports the use of starch-based hydrogel systems for sustained, localized chemotherapeutic delivery and/or reduced systemic exposure. The extended plasma half-life and low relative bioavailability confirm that the formulation permits controlled drug release at the injection site while reducing peak systemic concentrations.

These findings support feasibility of hydrogel-based modulation of doxorubicin pharmacokinetics for improved safety and efficacy in solid tumor contexts.

In an example, the hydrogel formulation described in the doxorubicin pharmacokinetic study is extended to encapsulate gamma-emitting and alpha-emitting radionuclides, e.g., cerium-139 (¹³⁹Ce), cerium-144 (¹⁴⁴Ce), and/or actinium-225 (²²⁵Ac). The compatibility of the starch-based hydrogel with small-molecule chemotherapeutics and metal-based radiotherapeutics depends at least in part on its high internal surface area, tunable porosity, and capacity for swelling-mediated uptake. These features enable the hydrogel to retain diverse payloads and promote controlled diffusion under physiological conditions. In some embodiments, the hydrogel matrix supports loading of radiometals under mild thermal and/or mechanical conditions and provides release profiles suitable for tumor-localized radionuclide therapy. The chemotherapeutic and radionuclide encapsulation studies together establish the hydrogel as a modular platform for intratumoral therapy combining DNA-damaging agents and/or targeted radiopharmaceuticals.

In some embodiments, the hydrogel composition enables localized drug retention while minimizing systemic exposure, as evidenced by the pharmacokinetic data. The plasma concentration of doxorubicin may decrease rapidly after administration and may fall below 10 μg/L within 4 to 6 hours. This systemic clearance behavior may be attributable to sustained local release, limited vascular diffusion, and the depot-like behavior of the starch-based hydrogel. The presence of a prolonged low-concentration terminal phase suggests minimal systemic redistribution or recirculation. These characteristics enhance safety by reducing the likelihood of systemic toxicity associated with doxorubicin. The low systemic levels over 24 hours may also be advantageous in pediatric or immunocompromised patient populations and may demonstrate a non-obvious benefit over conventional formulations lacking a hydrogel carrier.

Pharmacokinetic analysis of doxorubicin (DOX) following administration of a hydrogel composition to a tumor-bearing subject demonstrates rapid systemic clearance and sustained local retention. Plasma concentration measurements obtained using liquid chromatography-tandem mass spectrometry (LC-MS/MS) from samples collected at predetermined intervals post-injection reveal initial DOX levels exceeding 100 μg/L at 0.25 hours, declining rapidly to <10 μg/L by approximately 6 hours, followed by a plateau phase extending through 24 hours. The concentration remains above the lower limit of quantification (LLOQ) of approximately 0.1 ug/L throughout the monitoring period. Mean values from multiple biological replicates confirm time-dependent drug clearance from systemic circulation with a characteristic tri-phasic disposition profile. The plasma concentration spans four orders of magnitude from peak to terminal levels, demonstrating effective local retention of the therapeutic agent and minimal systemic leakage. This pharmacokinetic profile supports the novelty of sustained, localized drug release with reduced systemic exposure enabled by the hydrogel formulation, distinguishing it from conventional free drug administration.

In some embodiments, the syringe is manufactured under aseptic conditions and/or filled after terminal sterilization of the hydrogel composition. The sterilization may comprise gamma irradiation, steam sterilization, and/or a combination thereof. The syringe may comprise a transparent barrel that enables visual inspection and/or fluorescence-based detection of the hydrogel composition prior to administration. The syringe may comprise a machine-readable identifier, e.g., a barcode or data matrix code, that enables batch tracking and/or dose traceability. The syringe may be integrated into image-guided administration workflows using ultrasound, magnetic resonance imaging (MRI), and/or other real-time visualization methods. The syringe may comprise labeling that indicates composition details, dosing information, and/or administration instructions.

A pre-filled hydrogel injection system comprises a syringe loaded with a pre-formed hydrogel targeted for delivery into a solid tumor. The syringe contains a stable single-phase hydrogel retained in a sealed container, with cold chain storage requirements for maintaining formulation stability. Thermal warning indicators and single-use labeling denote proper storage and administration protocols to ensure therapeutic efficacy and safety. The hydrogel is delivered directly into the tumor using a standard needle-based method compatible with image-guided injection techniques. The pre-filled system demonstrates the ability to maintain pre-formed hydrogel integrity through handling and storage while enabling precise site-specific delivery. This configuration eliminates the need for bedside preparation or reconstitution, supporting streamlined clinical workflows and reducing the potential for dosing errors. The sealed syringe format provides protection from environmental contamination and enables standardized dose delivery across multiple treatment sites. The injection system is compatible with standard medical imaging modalities and surgical approaches, facilitating integration into existing oncological treatment protocols.

In some embodiments, the hydrogel composition is administered intratumorally to a pediatric solid tumor, such as a brain tumor, to suppress tumor progression and reduce systemic exposure. The composition may comprise a therapeutic agent selected for localized retention and diffusion within tumor tissue. The composition may promote sustained suppression of tumor growth as compared to untreated controls and may be compatible with stereotactic delivery techniques. In some embodiments, the hydrogel composition is evaluated using orthotopic models that replicate anatomical and pathological features of pediatric brain tumors, and/or together with longitudinal imaging and measurement of tumor volume to monitor efficacy. The composition may comprise a starch-based hydrogel delivering a chemotherapeutic or radionuclide agent and/or a combination thereof.

A representative evaluation of local tumor suppression using the hydrogel composition demonstrates significant therapeutic efficacy in preclinical models. Tumor growth analysis over 21 days shows that treated groups display markedly attenuated tumor progression compared to untreated controls, with relative tumor volume measurements confirming sustained anti-tumor activity. The hydrogel composition achieves substantial reduction in tumor burden through localized therapeutic delivery, avoiding the systemic toxicities associated with conventional chemotherapy regimens. Delivery methodology involves direct intratumoral injection of the hydrogel composition into pediatric brain tumors using standard neurosurgical approaches, with the syringe introduced through minimally invasive access points in the cranial anatomy. The treatment protocol demonstrates compatibility with pediatric neurosurgical procedures and supports clinical application in challenging anatomical locations. This localized delivery approach enables high-concentration therapeutic exposure at the tumor site while minimizing systemic distribution, making it particularly suitable for pediatric patients where systemic toxicity concerns are heightened. The sustained tumor suppression observed over the monitoring period supports the clinical potential of the hydrogel composition in pediatric neurosurgical oncology applications . . .

A gamma imaging process following intratumoral injection of a diagnostic radionuclide-labeled hydrogel enables real-time visualization and monitoring of therapeutic delivery. The hydrogel formulation comprising a diagnostic radionuclide is delivered via syringe injection into a solid tumor mass, where emitted gamma radiation from the encapsulated radionuclide provides detectable signals for external imaging systems. Gamma camera detection arranged externally enables non-invasive imaging of the composition without requiring invasive monitoring procedures. The system demonstrates isotropic diffusion of the labeled payload within the tumor volume and directional transmission of gamma rays for optimal signal acquisition and spatial resolution. This imaging-enabled hydrogel delivery configuration supports integration of diagnostic functions with locoregional treatment, allowing clinicians to confirm accurate placement, monitor therapeutic distribution, and assess retention kinetics in real-time. The dual functionality eliminates the need for separate diagnostic and therapeutic procedures, streamlining clinical workflows while providing quantitative feedback on treatment delivery. This approach supports integration of diagnostic and therapeutic functions using a single injectable hydrogel platform, enabling theranostic applications that combine localized therapy with immediate imaging confirmation of successful depot formation and spatial coverage within the target tumor volume.

In some embodiments, the composition comprises a crosslinked hydrogel matrix comprising dispersed chemotherapeutic agents, diagnostic moieties, and radionuclides. The matrix may support co-delivery of multiple therapeutic and diagnostic payloads without phase separation and/or agglomeration. The crosslinked network may be formed from starch-derived polymers that provide mechanical stability and compatibility with each embedded component. The chemotherapeutic agents may comprise an anthracycline and/or another cytotoxic compound. The radionuclide component may comprise an alpha-emitting or beta-emitting radionuclide. The diagnostic moiety may enable visualization using positron emission tomography (PET), single-photon emission computed tomography (SPECT), and/or fluorescence-based imaging. The components may be dispersed in a physically continuous solvent phase and may be retained in a single-phase composition suitable for local injection. The hydrogel may support prolonged depot formation and controlled release under physiological conditions. In some embodiments, the composition supports multimodal tumor localization and/or dual-mechanism therapeutic action.

A crosslinked hydrogel matrix comprising multiple embedded functional components demonstrates the modular design capability of the disclosed composition. The hydrogel matrix comprises polymer chains forming an interconnected network that provides structural integrity and controlled release properties. Distributed throughout the matrix are chemotherapeutic agents, which may comprise doxorubicin or structurally related compounds, enabling localized cytotoxic activity. Radionuclide particles are dispersed at distinct locations within the hydrogel and may correspond to beta- or alpha-emitting radionuclides suitable for therapeutic delivery, providing complementary radiotherapeutic mechanisms. Diagnostic imaging moieties are also present and enable real-time tracking using PET, SPECT, or optical imaging modalities, allowing for treatment monitoring and verification of depot placement. A continuous solvent phase is interspersed between the polymer strands, supporting molecular diffusion and maintaining mechanical integrity of the hydrogel under physiological conditions. The composition demonstrates relative distribution and spatial uniformity of all agents within a single gel matrix prior to injection, ensuring consistent therapeutic delivery. This multipayload loading within a single hydrogel phase supports the modular and combinatorial therapeutic design disclosed herein, enabling simultaneous delivery of chemotherapy, radiotherapy, and diagnostic capabilities through a single injection procedure while maintaining the stability and biocompatibility of each functional component.

In some embodiments, the hydrogel composition is applicable to a variety of tumor types. The tumor types may include but not limited to brain, breast, colorectal, pancreatic, hepatic, and/or sarcomatous tumors. The tumor may be malignant, invasive, and/or unresectable. The tumor may comprise a localized solid mass, nodular growth, an/or a combination thereof. The hydrogel composition may be formulated to deliver one or more therapeutic agents with activity against specific tumor subtypes, e.g., glioblastoma multiforme, medulloblastoma, triple-negative breast cancer, and/or pancreatic ductal adenocarcinoma. The composition may be administered to resected tumor margins, residual tumor beds, and/or unresected lesions. In some embodiments, the hydrogel is used in pediatric tumors such as brain tumors with high recurrence risk. The localized delivery and/or depot retention may provide therapeutic benefit in anatomically constrained tumor sites where systemic exposure is undesirable. The composition may be suitable for preclinical evaluation in xenograft, allograft, and/or orthotopic models of the aforementioned tumors. The composition may be deployed together with imaging-based outcome monitoring.

In some embodiments, the hydrogel composition is administered to pediatric tumors that are sensitive to local drug delivery and/or exhibit poor response to systemic chemotherapy. The tumor may comprise a medulloblastoma, ependymoma, and/or diffuse intrinsic pontine glioma. The tumor may be located within the central nervous system. The hydrogel composition may be injected during and/or after surgical resection of the tumor. The hydrogel may serve as a localized reservoir for sustained therapeutic delivery. The starch-based matrix may reduce systemic distribution and/or associated toxicities in pediatric patients. The matrix may support therapeutic efficacy in radiation- or drug-refractory disease. In some embodiments, the hydrogel is compatible with MRI-guided or stereotactic injection into deep brain regions. The composition may exhibit depot stability and/or therapeutic retention within the tumor site for at least about 7 days. The composition may suppress tumor recurrence in orthotopic pediatric tumor models. The composition may be formulated for single use, repeat administration, and/or a combination thereof.

In some embodiments, the hydrogel composition is administered as a single-dose or repeat-dose intratumoral formulation. The composition may be injected once to achieve sustained local retention and/or may be re-administered at defined intervals to maintain therapeutic exposure. The composition may be loaded with a therapeutic agent in a concentration selected to provide depot-mediated release over multiple days and/or weeks.

In repeat-dose regimens, the second administration may occur at least about 3 days, 5 days, 7 days, and/or 14 days after the initial injection. The composition may maintain injectable properties after storage and may be administered in a surgical, outpatient, or image-guided interventional setting. In some embodiments, repeat dosing may improve tumor suppression relative to single-dose controls and may be evaluated using survival analysis or tumor volume reduction metrics. The hydrogel may support re-injection without disrupting prior depot structure and may distribute uniformly across tumor volume during each administration.

In some embodiments, the hydrogel composition is delivered intratumorally using an image-guided technique selected to localize the injection within a tumor volume. The injection may be guided using magnetic resonance imaging (MRI), computed tomography (CT), ultrasound, and/or a combination thereof. The injection pathway may be selected based on tumor morphology, proximity to vital structures, and/or previous resection margins. The hydrogel composition may comprise a diagnostic radionuclide or imaging agent that permits real-time visualization and/or post-injection confirmation of distribution. The imaging modality may support localization of the hydrogel within the tumor, visualization of therapeutic coverage, and/or monitoring of degradation or clearance over time. In some embodiments, the hydrogel is co-formulated with a gamma-emitting or positron-emitting tracer and is visualized using gamma scintigraphy, single photon emission computed tomography (SPECT), or positron emission tomography (PET). Image-guided delivery may enhance therapeutic precision, reduce off-target exposure, and support personalized dosing regimens.

In some embodiments, the hydrogel composition comprises both a chemotherapeutic agent and a radionuclide selected to provide complementary modes of tumor cell killing. The chemotherapeutic agent may comprise doxorubicin, cisplatin, and/or another cytotoxic compound, and the radionuclide may comprise cerium-144, actinium-225, and/or a beta- or alpha-emitting radionuclide. The two agents may be co-encapsulated within the hydrogel matrix without chemical interference or phase separation. The chemotherapeutic agent may promote intercalation-induced cytotoxicity while the radionuclide provides ionizing radiation to damage nearby tumor cells. The co-formulated hydrogel may be injected intratumorally and may enable synergistic tumor suppression relative to either agent alone. In some embodiments, the composition is evaluated using fluorescence and radiometric tracking of the respective payloads in vivo and may demonstrate durable retention, tumor-localized cytotoxicity, and minimized systemic leakage. The multi-agent composition may address heterogeneous tumor populations and/or promote tumor microenvironment modulation while reducing off-target exposure.

In some embodiments, the hydrogel composition further comprises an imaging agent to support post-injection visualization, biodistribution analysis, and/or treatment verification. The imaging agent may comprise a gamma-emitting radionuclide, a positron-emitting tracer, a fluorescent dye, and/or a magnetic resonance contrast material. The imaging agent may be stably retained within the hydrogel matrix and may co-localize with therapeutic agents during and after administration. The imaging signal may be used to confirm complete delivery of the hydrogel, monitor spatial retention over time, and evaluate release kinetics within the tumor environment. In some embodiments, the imaging agent enables dual or multimodal imaging, e.g., combining fluorescence imaging with nuclear medicine techniques. The presence of an imaging component may allow for real-time feedback during surgical or interventional procedures and/or longitudinal assessment of treatment response. The imaging functionality may be added without compromising mechanical integrity, injectability, or therapeutic performance of the hydrogel composition.

In some embodiments, the hydrogel composition forms a localized therapeutic depot within the tumor site and reduces systemic distribution of the therapeutic agent. The depot may remain at the injection site under physiological conditions and may release the therapeutic payload over a defined period, e.g., at least about 3 days, 5 days, 7 days, and/or 14 days. The composition may prevent leakage of chemotherapeutic or radiotherapeutic agents into surrounding tissue and/or circulation and may reduce systemic toxicity relative to equivalent free-drug administration. In some embodiments, plasma pharmacokinetic analysis demonstrates that systemic levels of the therapeutic agent fall below detectable limits within 24 hours of administration, while therapeutic levels persist in the tumor for a longer duration. The depot effect may be enhanced by the viscoelasticity, crosslinking density, and solvent retention of the starch-based hydrogel. The localized retention may support higher intratumoral drug concentrations, improved tumor cell killing, and reduced adverse events.

In some embodiments, the hydrogel composition is co-administered with a systemic therapeutic agent to enable combination treatment of primary and metastatic disease. The hydrogel may be injected locally into the tumor site while a separate agent is administered intravenously, orally, and/or via another systemic route. The systemic agent may comprise an immune checkpoint inhibitor, targeted therapy, chemotherapeutic, or radiosensitizer. The hydrogel-based local therapy may debulk the primary tumor or deliver radiation locally, while the systemic agent addresses micrometastatic or circulating tumor cells. In some embodiments, the composition may be co-administered with boron-containing compounds for boron neutron capture therapy (BNCT), with the hydrogel delivering structural or tracer payloads. The combined regimen may be designed to enhance overall efficacy, prolong progression-free survival, and/or reduce dose-limiting toxicities compared to monotherapy. The timing, sequencing, and dose of each component may be adjusted based on tumor response and/or imaging-based feedback.

In some embodiments, a method of treating a solid tumor comprises administering a hydrogel composition intratumorally to a subject in need thereof. The composition comprises a starch-based polymer matrix, a solvent comprising dimethyl sulfoxide (DMSO), and a therapeutic agent comprising a chemotherapeutic compound, a radionuclide, and/or a combination thereof. The composition is prepared in advance of administration and is injected into the tumor mass using a syringe or image-guided delivery system. The injected hydrogel forms a localized depot that retains the therapeutic agent within the tumor volume for a defined duration. The method may be applied to treat malignant tumors located in the brain, breast, pancreas, colon, or other tissue sites. The hydrogel may promote localized cytotoxicity while minimizing systemic drug distribution. The method may be used as a standalone locoregional therapy or in combination with surgical resection, systemic treatment, or radiation therapy.

In some embodiments, the method comprises monitoring the therapeutic effect and biodistribution of the hydrogel composition following intratumoral administration. The tumor response may be assessed by imaging modalities such as magnetic resonance imaging (MRI), ultrasound, computed tomography (CT), and/or positron emission tomography (PET). The therapeutic effect may comprise reduction in tumor volume, attenuation of growth rate, and/or prolongation of survival. Systemic exposure may be evaluated using pharmacokinetic sampling and/or radiometric assays, including blood draws, scintigraphy, and gamma counting. In some embodiments, plasma concentrations of the therapeutic agent are below detectable limits at 24 hours after injection, while tumor-localized concentrations remain detectable for at least about 3 days, 5 days, and/or 7 days. The monitoring step may further comprise histological evaluation of the tumor and surrounding tissue to assess therapeutic penetration, necrosis, and agent retention within the hydrogel depot.

In some embodiments, the method comprises administering the hydrogel composition during or after a surgical or interventional oncology procedure. The composition may be injected into a tumor cavity following partial or complete surgical resection and may serve as a depot to eliminate residual tumor cells. The injection may be performed intraoperatively under direct visualization or in a post-operative setting using stereotactic or image-guided techniques. The hydrogel may conform to the geometry of the resection margin and may release therapeutic agents over time while remaining localized within the surgical site. In some embodiments, the method is applied to tumors for which complete resection is not feasible or where microscopic residual disease poses a recurrence risk. The hydrogel may reduce the need for immediate systemic chemotherapy and may improve local control while minimizing exposure to adjacent critical structures.

In some embodiments, the method comprises administering the hydrogel composition to a subject having a tumor located in a delicate or anatomically constrained region. The tumor may comprise a pediatric brain tumor, a spinal cord lesion, a brainstem glioma, and/or a tumor located near critical vasculature or neural structures. The hydrogel may be injected using a fine-gauge needle and/or image-guided approach selected to minimize collateral tissue damage. The depot formed by the composition may remain localized to the injection site and may release therapeutic agents in a controlled manner. The composition may be formulated to reduce viscosity during injection and may be retained in gelled form following administration. In some embodiments, the method is used to avoid systemic administration of cytotoxic agents in pediatric subjects and/or in cases where systemic chemotherapy is contraindicated. The hydrogel may support targeted treatment while maintaining anatomical and functional integrity of the surrounding tissue.

In some embodiments, the method comprises administering a hydrogel composition comprising a diagnostic imaging agent to enable visualization of depot formation and therapeutic localization. The imaging agent may comprise a radionuclide detectable by positron emission tomography (PET), single photon emission computed tomography (SPECT), and/or a fluorescent or optically active tracer. The imaging agent may be co-loaded with a therapeutic agent and may be retained within the hydrogel following administration. The imaging signal may be acquired immediately after injection to confirm depot placement and/or at subsequent time points to monitor retention, degradation, or diffusion. In some embodiments, the method comprises acquiring a baseline scan, administering the hydrogel, and performing follow-up scans at defined intervals. The imaging signal may support adaptive therapeutic planning and/or confirm complete coverage of the tumor volume. The method may further comprise co-registration of imaging data with anatomical imaging for composite interpretation.

In some embodiments, the method comprises administering the hydrogel composition as a repeatable intratumoral treatment based on therapeutic response or imaging feedback. The composition may be injected once to establish a therapeutic depot and may be re-administered after a defined interval based on tumor size, retention, and/or imaging signal intensity. The interval between administrations may be at least about 3 days, 5 days, 7 days, and/or 14 days. The composition may be re-injected at the same site, adjacent regions, and/or across multiple tumor loci. In some embodiments, repeat administration is performed under image guidance and may involve adjustment of injection volume or composition based on prior retention patterns. The method may support dose escalation, adaptive therapy, and/or tailored delivery based on subject-specific tumor characteristics. The hydrogel may retain physical integrity during each administration and may distribute uniformly without disrupting previously delivered material.

In some embodiments, the method comprises treating a solid tumor using a hydrogel composition comprising multiple functional components that act through distinct mechanisms. The composition may comprise a chemotherapeutic agent that promotes apoptosis and/or DNA intercalation, a radionuclide that delivers cytotoxic radiation, and/or an imaging agent that enables spatial tracking. The method may comprise a single intratumoral injection of the hydrogel followed by local depot formation and sustained release of the therapeutic agents. In some embodiments, the hydrogel simultaneously releases a chemotherapeutic compound and emits localized radiation, producing synergistic anti-tumor effects. The imaging component may enable real-time confirmation of delivery and longitudinal monitoring of depot integrity. The method may be used in anatomically challenging tumors where multimodal therapy is preferred and/or where systemic delivery is contraindicated. The hydrogel composition may promote additive or synergistic tumor cell killing through complementary modes of action.

In some embodiments, a method of forming a hydrogel composition comprises mixing a starch-based polymer with a solvent and a therapeutic agent under ambient or cooled conditions. The starch-based polymer may comprise amylose, amylopectin, and/or a starch-derived polysaccharide blend having a defined molecular weight distribution. The solvent may comprise dimethyl sulfoxide (DMSO) and/or a polar aprotic solvent that promotes swelling and dispersion of the polymer matrix. The therapeutic agent may comprise a chemotherapeutic compound, a radionuclide, and/or a combination thereof. The components may be mixed in a sterile container using a vortex, spatula, or automated mixer. The resulting mixture may form a single-phase hydrogel with viscoelastic properties suitable for injection. The order of addition may depend at least in part on the solubility and stability of the therapeutic agent. In some embodiments, the therapeutic agent is dissolved in the solvent prior to addition to the starch polymer.

In some embodiments, the method further comprises crosslinking the starch polymer matrix using a multifunctional crosslinking agent. The crosslinking agent may comprise d-glucaric acid and/or another molecule comprising at least two carboxylic acid or hydroxyl functional groups. The crosslinker may be added to the starch solution under controlled pH conditions, e.g., between about pH 3.0 and pH 6.5, and/or at a temperature of at least about 20° C., 25° C., and/or 30° C. The crosslinking reaction may be allowed to proceed for at least about 10 minutes, 20 minutes, and/or 30 minutes before hydrogel stabilization. The degree of crosslinking may be adjusted by modulating the concentration of the crosslinker and/or the duration of the reaction. The resulting matrix may exhibit a storage modulus that supports depot formation and retention in vivo. The crosslinked hydrogel may resist dissolution in aqueous media and may maintain its physical integrity during injection and storage.

In some embodiments, the hydrogel composition provides a localized depot for sustained delivery of therapeutic agents at the tumor site while minimizing systemic exposure. The depot may retain the therapeutic payload under physiological conditions and may maintain its structural integrity for a defined period after intratumoral injection. The starch-based matrix may promote viscoelastic behavior and swelling that enable retention within the tumor microenvironment. The depot effect may result in higher local concentrations of chemotherapeutic or radiotherapeutic agents relative to systemic delivery, thereby enhancing efficacy and reducing off-target toxicity. In some embodiments, pharmacokinetic analysis confirms that systemic drug levels fall below detection limits within 24 hours, while intratumoral levels remain elevated for at least about 3 days, 5 days, and/or 7 days. The depot may be visible by imaging and may support real-time confirmation of successful injection.

In some embodiments, the hydrogel composition enables co-encapsulation of multiple functional agents without requiring chemical conjugation or separate delivery vehicles. The composition may comprise a chemotherapeutic compound, a radionuclide, and/or an imaging agent, each retained within the matrix via physical entrapment or non-covalent interaction. The co-formulation allows simultaneous delivery of cytotoxic and radiotherapeutic mechanisms while permitting visualization of depot placement and agent distribution. The multi-agent system may reduce the number of injections required, simplify dosing regimens, and support synergistic tumor cell killing. The composition may maintain encapsulation efficiency and uniformity across all payloads during manufacturing, storage, and administration. In some embodiments, agent release kinetics may be tuned through matrix formulation or crosslinking density, allowing coordinated or sequential therapeutic effects. The hydrogel may eliminate the need for separate carriers, chelators, or multiple devices, thereby reducing complexity and improving clinical workflow.

In some embodiments, the hydrogel composition offers adaptability across a range of tumor types, anatomical locations, and clinical settings. The composition may be injected into resectable or unresectable tumors, superficial or deep-seated lesions, and/or tumors located in delicate tissues such as the brain, spine, or pediatric sites. The hydrogel may be administered via fine-gauge needles compatible with stereotactic or image-guided procedures and may conform to the geometry of complex tumor cavities. The matrix may exhibit shear-thinning behavior during injection and may return to a gelled state in situ, supporting accurate placement and retention. The hydrogel formulation may be customized based on tumor volume, proximity to critical structures, and therapeutic goals. In some embodiments, the composition may be used intraoperatively, perioperatively, or in outpatient interventional workflows. The adaptability of the platform may support expansion to diverse patient populations and tumor indications.

In some embodiments, the hydrogel composition and its method of manufacture support scalability, reproducibility, and compatibility with clinical workflows. The formulation may be prepared using pharmaceutically acceptable components under ambient or mildly elevated conditions without requiring specialized equipment. The composition may be sterilized using gamma irradiation, aseptic mixing, and/or sterile filtration depending at least in part on the therapeutic agent. The hydrogel may be loaded into pre-filled syringes using standard fill-finish processes and may be stored for at least about 2 weeks, 4 weeks, and/or 6 weeks without loss of function. The pre-filled configuration may support point-of-care use, intraoperative injection, and rapid field deployment. In some embodiments, the hydrogel passes through 25G, 27G, and/or 30G needles without clogging or loss of depot-forming capability. The composition may be compatible with existing diagnostic imaging infrastructure and may reduce procedural burden relative to conventional implantable systems.

In some embodiments, the hydrogel composition enhances treatment safety, precision, and adaptability by integrating therapeutic and diagnostic functionality into a single injectable platform. The localized delivery reduces systemic exposure and may decrease dose-limiting toxicities associated with free drug or radiopharmaceutical administration. The inclusion of imaging agents may allow for confirmation of complete depot formation, early detection of misplacement, and longitudinal tracking of therapeutic retention. In some embodiments, the composition supports personalized treatment planning, including dose adjustment and retreatment based on imaging feedback. The co-delivery of multiple agents may increase therapeutic efficacy without requiring multiple injections or systemic dosing regimens. The modularity of the formulation allows clinicians to select payload combinations appropriate for tumor type, patient characteristics, and treatment goals. The integration of safety, tracking, and precision targeting may enable broader use of locoregional therapies in challenging tumor settings.

In some embodiments, the hydrogel composition is used as a local delivery platform for encapsulated radionuclides, supporting translation as a combination product or drug-device system under applicable regulatory frameworks (e.g., 505 (b) (2) or EU MDR Article 117). The pre-gelled starch-based hydrogel enables syringe-compatible injection of radionuclide-loaded matrices (e.g., ¹⁴⁴Ce, ²²⁵Ac) into tissue sites, such as tumor margins or post-resection beds, to achieve sustained therapeutic irradiation. Encapsulation protocols optimized at 40° C. and 7500 rpm for 30 minutes with ≤1 μL of CeAccPro (˜400 nCi) have demonstrated >80% encapsulation efficiency and reduced off-target diffusion over 48 to 168 hours (e.g., Examples 2400-3500). These characteristics align with the FDA's expectations for local drug retention and safety margin control in radiotherapeutic delivery. The hydrogel's formulation and excipient components (e.g., DMSO, amylopectin) are GRAS-listed and may be supported by prior device master files (e.g., for syringe systems), facilitating preclinical planning and regulatory alignment. In some embodiments, the composition may be adapted for loading with α-emitting radionuclides (e.g., ²²⁵Ac), enabling modular extension to next-generation radiotherapies.

In some embodiments, a starch-based hydrogel comprising amylopectin and dimethyl sulfoxide (DMSO) is evaluated using cerium (III) chloride hexahydrate (CeCl₃·6H₂O) as a surrogate for radiotherapeutic payloads. The hydrogel demonstrates encapsulation efficiency of at least about 80% under dynamic conditions (e.g., 40° C., 15000 revolutions per minute (rpm), 30 minutes), and retains the surrogate cation under aqueous challenge for up to 90 minutes. Ultraviolet-visible spectroscopy (UV-Vis) and Fourier-transform infrared spectroscopy (FTIR) confirm encapsulation and solvent exchange dynamics. The use of CeCl₃ as a non-radioactive analog provides a predictive model for therapeutic radionuclide handling in good manufacturing practice (GMP)-compliant environments and may be integrated into regulatory filings governed by Title 21 of the Code of Federal Regulations (21 CFR), including Part 312 (Investigational New Drug Applications).

In some embodiments, the starch-based hydrogel loaded with CeCl₃ surrogate demonstrates compatibility with actinium-based alpha emitters such as actinium-225 (225Ac), which are under investigation for pediatric neuroblastoma. The low-temperature, small-volume encapsulation protocol minimizes matrix disruption, enabling safe delivery to tumor margins in sensitive patient populations. The surrogate model supports filing under the U.S. Pediatric Research Equity Act (PREA) or the European Medicines Agency (EMA) Pediatric Investigation Plan (PIP).

In some embodiments, the CeCl₃ surrogate-loaded hydrogel is representative of trivalent lanthanides suitable for intranodal administration. The hydrogel matrix demonstrates sustained release kinetics and biocompatibility in aqueous environments, making it suitable for immunomodulatory or radiotherapeutic delivery to lymphatic structures. The surrogate data support development of a drug-device combination product under European Union (EU) Medical Device Regulation (MDR) 2017/745, Article 117.

In some embodiments, the encapsulation and retention of CeCl₃ by the hydrogel enables modeling of bone-seeking radionuclides such as samarium-153 (¹⁵³Sm) or thorium-227 (²²⁷Th). The ability to retain over 75% of the loaded cationic species over 90 minutes highlights the potential for targeted delivery in skeletal metastases. Regulatory pathways may include Fast Track or Breakthrough Therapy designation under the U.S. Food and Drug Administration (FDA) if used with radionuclides for metastatic castration-resistant prostate cancer (mCRPC).

In some embodiments, the starch-based hydrogel is loaded with radioactive cerium radionuclides (e.g., cerium-144 (¹⁴⁴Ce)) under optimized dynamic conditions (e.g., 40° C., 7500 rpm, 30 minutes). Radiolabeling efficiency exceeds 90%, and retention is confirmed by γ-ray spectroscopy. The hydrogel matrix retains activity over extended periods (e.g., 48 to 168 hours), confirming suitability for use in targeted radiotherapeutic (TRT) applications. These data demonstrate compliance with regulatory guidance under United States Pharmacopeia (USP) General Chapter <825> and FDA expectations for radiopharmaceuticals used in diagnostic or therapeutic settings.

In some embodiments, the hydrogel is used to co-encapsulate therapeutic and diagnostic radionuclides (e.g., ¹⁴⁴Ce for therapeutic payloads and ¹³⁹Ce for imaging calibration). The observed release kinetics and radiolabeling performance enable use in theranostic platforms aligned with Clinical Trials Regulation (EU) No 536/2014 or FDA guidance for diagnostic radiopharmaceuticals.

In some embodiments, the hydrogel platform enables local delivery of radionuclides to intracranial tumor sites. The encapsulation and retention characteristics observed with ¹⁴⁴Ce are predictive of behavior with α-emitting radionuclides such as ²²⁵Ac, relevant to glioblastoma multiforme treatment strategies. This supports expanded access or orphan drug designation pathways under 21 CFR Part 316. In some embodiments, radionuclide-loaded hydrogels offer localized delivery to bone or soft tissue sites in prostate cancer patients. The DMSO-based hydrogel matrix supports microvolume radionuclide loading and extended retention under physiological conditions.

The present disclosure contemplates that the terminology used herein is intended to describe particular examples and is not limiting. The terms “comprise,” “comprises,” and “comprising” are inclusive and are intended to cover elements that include, but are not limited to, the stated components. The term “may” expresses optionality and does not imply requirement. The phrase “and/or” is used to reflect that one or more of the listed elements may be present, either individually or together.

The present disclosure contemplates that any of the compositions, methods, devices, processes, and components described herein may be combined in any operable manner, unless explicitly stated to be incompatible. Structural features, therapeutic payloads, delivery formats, imaging options, and radiotherapeutic properties may be incorporated in any combination that supports the intended technical effect.

The present disclosure contemplates that structural or functional variants equivalent to those described may also fall within the scope of the invention. Such variants may include analogs, derivatives, isotopologues, homologues, prodrugs, or other pharmacologically acceptable substitutes of the disclosed therapeutic agents, solvents, radionuclides, and/or excipients.

The present disclosure contemplates that the examples, figures, and photographic images are illustrative and are not intended to limit the claimed subject matter. Compositions and processes shown in graphical or photographic form may reflect specific embodiments and may be varied or substituted while remaining within the teachings of the disclosure.

The present disclosure contemplates that support is provided for product claims, method of use claims, method of manufacture claims, apparatus and system claims, and non-transitory computer-readable media claims. This includes claims directed to hydrogel compositions, injection workflows, depot behavior, and imaging-feedback-based scheduling.

The present disclosure contemplates that use-based claim formats may be supported for treatment of specific tumor types, anatomical regions, subject populations, or surgical settings. This may include adult and pediatric patients, resectable or unresectable tumors, and post-operative therapeutic delivery.

The present disclosure contemplates that technical terminology used to describe chemical, biological, and clinical effects should be interpreted consistent with the understanding of a skilled artisan in drug delivery, oncology, polymer chemistry, and/or interventional radiology. Terms relating to injection, imaging, and radiation should be construed according to conventional technical usage.

The present disclosure contemplates that headings and figure references are provided for organizational clarity and do not affect claim scope. Paragraph numbers, figure order, and image labels do not imply sequential dependency or technical limitation unless explicitly described. Each paragraph and figure is independently combinable and claim-enabling.

The present disclosure contemplates that variations in terminology, figure labeling, or example formatting do not affect the scope or construction of the claims. Technical effects, features, and compositions described under different naming conventions may refer to equivalent structures or embodiments, and such terms should be interpreted contextually based on the function described.

The present disclosure contemplates that equivalents of the described features may include structural, chemical, mechanical, and/or biological substitutions that achieve substantially similar results. Such equivalents may include hydrogel matrices formed using alternative polysaccharides, crosslinkers with analogous functionality, and/or radionuclides with similar emission characteristics, so long as the technical effect described is retained.

The present disclosure contemplates that the teachings herein may be applied to emerging therapeutic settings, diagnostic strategies, and/or manufacturing technologies. This includes incorporation into automated formulation systems, image-guided injection platforms, and/or hybrid devices that combine therapeutic delivery with diagnostic feedback. Support is provided for integration with existing treatment algorithms or clinical standards of care.

While the disclosure is described in terms of certain embodiments, structures, and operations, those skilled in the art will recognize that variations, modifications, and equivalents can be made without departing from the scope of the present disclosure. The compositions, systems, devices, and methods described herein may be practiced in various combinations, configurations, or sequences as appropriate. The examples, figures, and data presented herein are provided to support enablement and illustration and are not intended to limit the scope of the appended claims.

### REFERENCES

Each of the following references are hereby incorporated herein in their entirety:
1. K. A. Cronin et al., “Annual Report to the Nation on the Status of Cancer, part I: National cancer statistics,” Cancer, vol. 124, no. 13, 2018, doi: 10.1002/cncr.31551.
2. R. L. Siegel, A. N. Giaquinto, and A. Jemal, “Cancer statistics, 2024,” CA: A Cancer Journal for Clinicians, vol. 74, no. 1, 2024, doi: 10.3322/caac.21820.
3. S. N. Garima Mathur, Pramod Kumar Sharma “Cancer: An Overview,” Academic Journal vol. 2015, of Cancer Reserch, 8, no. 1, doi:

10.5829/idosi.ajcr.2015.8.1.9336.
4. C. Sawyers and C. Sawyers, “Targeted cancer therapy,” Nature, vol. 432, no. 7015, 2004, doi: 10.1038/nature03095.
5. V. V. Padma and V. V. Padma, “An overview of targeted cancer therapy,” BioMedicine, vol. 5, no. 4, 2015, doi: 10.7603/s40681-015-0019-4.
6. D. T. Debela et al., “New approaches and procedures for cancer treatment: Current perspectives,” SAGE Open Medicine, vol. 9, p. 20503121211034366, 2021, doi: 10.1177/20503121211034366.
7. C. Parker et al., “Targeted Alpha Therapy, an Emerging Class of Cancer Agents: A Review,” JAMA oncology, vol. 4, no. 12, pp. 1765-1772, 2018, doi: 10.1001/jamaoncol.2018.4044.
8. A. R. Ankit Verma, Vaishnavi Sahu, Yogendra Chaurasia, Amit Kumar, Swati Rathore, Richa Tripathi et al., “Current advances in cancer treatment: A comprehensive review of therapeutic strategies and emerging innovations,” World Journal of Biology Pharmacy and Health Sciences, vol. 18, no. 1, 2024-0423T16:28:07+05:30, doi: 10.30574/wjbphs.2024.18.1.0204.
9. “Injectable polypeptide micelles that form radiation crosslinked hydrogels in situ for intratumoral radiotherapy,” Journal of Controlled Release, vol. 228, 2016, doi: 10.1016/j.jconrel.2016.02.040.
10.C. C. Yanyan Hu, Shunhao Wang, Lingxiong Wang, Ping Liang, Fangyi Liu, Wenting Shang, Weiwei Wang, Fengrong Zhang, Shanshan Li, Heyun Shen, Xiaoling Yu, Huiyu Liu, Jie Tian, “A Comparative Study of Clinical Intervention and Interventional Photothermal Therapy for Pancreatic Cancer,” Advanced Materials, vol. 29, no. 33, 2017, doi: 10.1002/adma.201700448.
11. X. Li, X. Xu, M. Xu, Z. Geng, P. Ji, and Y. Liu, “Hydrogel systems for targeted cancer therapy,” Frontiers in Bioengineering and Biotechnology, vol. 11, 2023, doi: 10.3389/fbioe.2023.1140436.
12. R. Narayanaswamy and V. P. Torchilin, “Hydrogels and their applications in targeted drug delivery,” Molecules (Basel, Switzerland), vol. 24, no. 3, p. 603, 2019, doi: 10.3390/molecules24030603.
13. K. Nagahama, D. Kawano, N. Oyama, A. Takemoto, T. Kumano, and J. Kawakami, “Self-Assembling Polymer Micelle/Clay Nanodisk/Doxorubicin Hybrid Injectable Gels for Safe and Efficient Focal Treatment of Cancer,” Biomacromolecules, vol. 16, no. 3, 2015, doi: 10.1021/bm5017805.
14. L. Arazi et al., “Alpha DaRT: Revolutionary Alpha-Emitters Brachytherapy,” Journal of Medical Imaging and Radiation Sciences, vol. 50, no. 1, 2019, doi: 10.1016/j.jmir.2019.03.083.
15. L. Arazi et al., “Alpha DaRT: Revolutionary Alpha-Emitters Brachytherapy,” Journal of Medical Imaging and Radiation Sciences, vol. 50, no. 1, 2019 Mar. 1, doi: 10.1016/j.jmir.2019.03.083.
16. H. Confino et al., “Tumor ablation by intratumoral Ra-224-loaded wires induces anti-tumor immunity against experimental metastatic tumors,” Cancer Immunology, Immunotherapy 2014 64:2, vol. 64, no. 2, 2014 Oct. 18, doi: 10.1007/s00262-014-1626-8.
17. “Radiochemical separation of 224Ra from 232U and 228Th sources for 224Ra/212Pb/212Bi generator,” Applied Radiation and radionuclides, vol. 172, 2021 Jun. 1, doi: 10.1016/j.apradiso.2021.109655.
18. B. J. B. Nelson et al., “Theranostic Imaging Surrogates for Targeted Alpha Therapy: Progress in Production, Purification, and Applications,” Pharmaceuticals 2023, Vol. 16, Page 1622, vol. 16, no. 11, 2023, doi: 10.3390/ph16111622.
19. “Alpha-particles for targeted therapy,” Advanced Drug Delivery Reviews, vol. 60, no. 12, 2008 Sep. 15, doi: 10.1016/j.addr.2008.04.007.
20. “Initial Safety and Tumor Control Results From a “First-in-Human” Multicenter Prospective Trial Evaluating a Novel Alpha-Emitting Radionuclide for the Treatment of Locally Advanced Recurrent Squamous Cell Carcinomas of the Skin and Head and Neck,” International Journal of Radiation Oncology*Biology*Physics, vol. 106, no. 3, 2020 Mar. 1, doi: 10.1016/j.ijrobp.2019.10.048.
21. M. Pruszyński, R. Walczak, M. Rodak, F. Bruchertseifer, A. Morgenstern, and A. Bilewicz, “Radiochemical separation of 224Ra from 232U and 228Th 224Ra/212Pb/212Bi generator,” Applied Radiation and radionuclides, vol. 172, 2021, doi: 10.1016/j.apradiso.2021.109655.
22. E. D. Barker, “The Synthesis and Characterization of a Novel Polysaccharide Hydrogel for Biomedical Applications Including the Treatment of Malignant Tumors and the Prevention of Metastatic Disease,” Master of Science Thesis, Tickle College of Engineering, University of Tennessee, Knoxville, 2007.
23. E. D. Barker, “Preclinical Evaluation of a Novel Polysaccharied Hydrogel for Local Chemotherapy Delivery to Solid Tumors,” Doctor of Philosphy, Tickle College of Engineering, University of Tennessee, Knoxville, 2014.
24. J. Patel, “A Local Drug Delivery Approach for Enhancing Tissue Penetration of Chemotherapy to Improve Treatment Outcomes of Pediatric Brain Tumors,” Doctor of Philosphy, Tickle College of Engineering, University of Tennessee, Knoxville, 2021.
25. F. S. Rocha, A. J. Gomes, C. N. Lunardi, S. Kaliaguine, and G. S. Patience, “Experimental methods in chemical engineering: Ultraviolet visible spectroscopy-UV-Vis,” Canadian journal of chemical engineering, vol. 96, no. 12, pp. 2512-2517, 2018, doi: 10.1002/cjce.23344.
26. T. Owens, “Fundamentals of Modern UV-visible Spectroscopy: A Workbook,” 1st ed. Berlin, Germany: Hewlett Packard Company, 1998.
27. A. Dutta, “Fourier Transform Infrared Spectroscopy,” vol. 2. United States: Elsevier, 2017, pp. 73-93.
28. Y. Nir-El, “Correction for decay during counting in gamma spectrometry,” Radiation Protection Dosimetry, vol. 153, no. 3, 2013, doi: 10.1093/rpd/ncs106.
29. (2011). Gamma Spectroscopy Overview.
30. (2011). Gamma Spectrum Features.
31. A. Coletta et al., “Solvent Dependency of the UV-Vis Spectrum of Indenoisoquinolines: Role of Keto-Oxygens as Polarity Interaction Probes,” PLOS ONE, vol. 8, no. 9, 2013, doi: 10.1371/journal.pone.0073881.
32. H. L. Greenhaus, A. M. Feibush, and L. Gordon, “Ultraviolet Spectrophotometric Determination of Cerium (III),” (in English), Anal. Chem., Article vol. 29, no. 10, pp. 1531-1534, 1957, doi: 10.1021/ac60130a045.
33. C. Berthomieu, R. Hienerwadel, C. Berthomieu, and R. Hienerwadel, “Fourier transform infrared (FTIR) spectroscopy,” Photosynthesis Research 2009 101:2, vol. 101, no. 2, 2009 Jun. 10, doi: 10.1007/s11120-009-9439-x.
34. J. Ravi et al., “FTIR markers of methionine oxidation for early detection of oxidized protein therapeutics,” European Biophysics Journal 2011 40:3, vol. 40, no. 3, 201101-13, doi: 10.1007/s00249-010-0656-1.
35. N. Mozhzhukhina, L. P. Méndez De Leo, and E. J. Calvo, “Infrared Spectroscopy Studies on Stability of Dimethyl Sulfoxide for Application in a Li-Air Battery,” Journal of physical chemistry. C, vol. 117, no. 36, pp. 18375-18380, 2013, doi: 10.1021/jp407221c.
36. U. Reus and W. Westmeier, “Catalog of gamma rays from radioactive decay,” Atomic data and nuclear data tables, vol. 29, no. 1, pp. 1-192, 1983, doi: 10.1016/S0092-640X (83) 80015-1.

I/We claim:

1. A composition comprising:
a starch-derived polymer forming a hydrogel matrix;
a solvent comprising dimethyl sulfoxide, an aqueous solvent selected from phosphate buffered saline or sodium chloride solution, or a combination thereof; and
a chemotherapeutic agent comprising
doxorubicin in its free base form or
a compound that is insoluble in water and substantially soluble in dimethyl sulfoxide,
wherein the composition may be a pre-formed injectable hydrogel formulation comprising the therapeutic agent prior to administration.

2. The composition of claim 1, wherein the hydrogel comprises a crosslinking agent comprising d-glucaric acid; optionally wherein the starch-derived polymer comprises a mixture of high molecular weight and low molecular weight amylopectin species; optionally wherein the hydrogel is synthesized in a solvent system comprising dimethyl sulfoxide and water; and optionally wherein the hydrogel is synthesized without initiators or catalysts.

3. The composition of claim 1, wherein the hydrogel retains at least about 90% of the therapeutic agent under physiological conditions; optionally wherein the retention is maintained over a period of at least about 24 hours; optionally wherein the retention is determined by liquid chromatography, ultraviolet-visible spectroscopy, or plasma pharmacokinetic analysis; and optionally wherein the therapeutic agent comprises doxorubicin in its free base form.

4. The composition of claim 1, wherein the chemotherapeutic agent penetrates at least about 10 mm into surrounding tissue from an intratumoral injection site; optionally wherein the penetration depth is determined by fluorescence imaging or histological analysis;
optionally wherein the chemotherapeutic agent comprises doxorubicin in its free base form; and optionally wherein the composition is administered to a pediatric brain tumor.

5. The composition of claim 1, wherein systemic exposure to the chemotherapeutic agent is reduced relative to an equivalent intravenous dose; optionally wherein the systemic exposure is determined by liquid chromatography-mass spectrometry; and optionally wherein the chemotherapeutic agent comprises doxorubicin in its free base form.

6. The composition of claim 1, wherein the hydrogel is sterilized and stored in a pre-filled syringe suitable for intratumoral injection; optionally wherein the syringe is configured for single use; optionally wherein the syringe is compatible with image-guided delivery techniques; and optionally wherein the hydrogel is stable for a storage period of at least about four weeks.

7. The composition of claim 1, wherein the composition is configured for administration to a pediatric solid tumor; optionally wherein the pediatric solid tumor comprises a brain tumor; optionally wherein the brain tumor comprises a medulloblastoma or glioma; and optionally wherein the composition is administered during or after surgical resection.

8. The composition of claim 1, wherein the chemotherapeutic agent comprises a lipophilic compound; optionally wherein the lipophilic compound is doxorubicin in its free base form; optionally wherein the lipophilic compound has a water solubility of less than about 1 mg/mL at 25° C.; and optionally wherein the solvent system enables solubilization and sustained retention of the lipophilic compound without precipitation.

9. The composition of claim 1, wherein the hydrogel is pre-formed and stable for storage as an injectable formulation; optionally wherein the formulation is maintained as a gel throughout storage and administration; optionally wherein the hydrogel is stable for at least about four weeks; and optionally wherein no gelling, mixing, or reconstitution step is required prior to injection.

10. The composition of claim 1, wherein the starch-derived hydrogel comprises a boron-containing therapeutic agent suitable for boron neutron capture therapy; optionally wherein the boron-containing therapeutic agent comprises a carborane, a boronic acid, or a boronated phenylalanine analog; and optionally wherein the hydrogel is retained at a tumor site prior to neutron exposure.

11. The composition of claim 1, wherein the chemotherapeutic agent comprises a compound that is insoluble in water and soluble in dimethyl sulfoxide; optionally wherein the agent comprises a topoisomerase inhibitor or an anthracycline compound.

12. A composition comprising:
a starch-based hydrogel comprising a polymer matrix;
a solvent comprising dimethyl sulfoxide, an aqueous solvent selected from phosphate buffered saline or sodium chloride solution, or a combination thereof; and
a radionuclide selected from
an alpha-emitting radionuclide,
a beta-emitting radionuclide, or
a gamma-emitting radionuclide,
wherein the composition is a pre-formed injectable hydrogel formulation comprising the radionuclide prior to administration.

13. The composition of claim 12, wherein:
the alpha-emitting radionuclide is selected from actinium-225, astatine-211, bismuth-213, lead-212, radium-223, terbium-149, thorium-227, thorium-226, fermium-225, or uranium-230;
the beta-emitting radionuclide is selected from lutetium-177, yttrium-90, samarium-153, rhenium-186, strontium-89, iodine-131, or phosphorus-32; and
the gamma-emitting radionuclide is selected from technetium-99m, indium-111, iodine-123, iodine-124, gallium-67, gallium-68, thallium-201, cobalt-57, krypton-81m, or xenon-133.

14. The composition of claim 12, wherein the hydrogel retains at least about 90% of the radionuclide upon formation; optionally wherein the encapsulation efficiency is determined by ultraviolet-visible spectroscopy or dialysis analysis; optionally wherein the radionuclide comprises cerium-144; and optionally wherein the starch polymer chains are crosslinked using d-glucaric acid.

15. The composition of claim 12, wherein the hydrogel retains at least about 90% of the radionuclide over a period of at least about 7 days under physiological conditions;
optionally wherein the retention is determined by dialysis or static diffusion testing;
optionally wherein the radionuclide comprises cerium-144 or cerium-141; and optionally wherein the hydrogel is maintained under aqueous conditions at 37° C. during testing.

16. The composition of claim 12, wherein the hydrogel is sterilized and stored in a pre-filled syringe suitable for intratumoral injection; optionally wherein the syringe is configured for single use and radiation-shielded storage; optionally wherein the composition remains physically stable in the syringe for at least about four weeks; and optionally wherein the syringe is compatible with image-guided delivery techniques.

17. The composition of claim 12, wherein systemic radioactivity resulting from administration of the composition is below detectable levels at 24 hours post-injection; optionally wherein the systemic radioactivity is assessed by gamma counting, scintigraphy, or radiometric detection of blood plasma; optionally wherein the radionuclide comprises cerium-144; and optionally wherein the composition is administered via intratumoral injection to a solid tumor.

18. The composition of claim 12, wherein the composition is configured for administration to a pediatric solid tumor; optionally wherein the pediatric solid tumor comprises a brain tumor; optionally wherein the brain tumor comprises a medulloblastoma or ependymoma; and optionally wherein the composition is administered locally during or after surgical resection of the tumor.

19. The composition of claim 12, wherein the radionuclide is selected from actinium-225, thorium-227, lutetium-177, or terbium-161; optionally wherein the radionuclide is selected based on its emission of alpha particles, beta particles, or Auger electrons; optionally wherein the composition comprises a chelator-free encapsulation of the radionuclide within the hydrogel matrix; and optionally wherein the composition is configured for site-specific delivery to a solid tumor.

20. The composition of claim 12, wherein the radionuclide comprises a gamma-emitting or positron-emitting radionuclide suitable for diagnostic imaging; optionally wherein the radionuclide comprises cerium-141, lutetium-177, or terbium-161; optionally wherein the composition enables detection by scintigraphy, PET, or SPECT imaging; and optionally wherein the composition is used to monitor intratumoral distribution following injection.

21. The composition of claim 12, wherein the hydrogel is physically and chemically stable for at least about four weeks; optionally wherein the radionuclide remains encapsulated without significant leakage during the storage period; optionally wherein the composition is maintained in a sealed container without requiring reconstitution prior to administration.

22. The composition of claim 12, wherein the radionuclide comprises actinium-225 and the composition emits visible luminescence due to Cherenkov radiation under storage or post-injection conditions; optionally wherein the luminescence is used to confirm physical retention of the radionuclide in the hydrogel.

23. The composition of claim 12, wherein the radionuclide is introduced into the hydrogel in the absence of a chelating agent; optionally wherein the composition provides in vivo retention of the radionuclide without use of diethylenetriaminepentaacetic acid (DTPA) or 1,4,7,10-tetraazacyclododecane-1,4,7,10-tetraacetic acid (DOTA).

24. A composition comprising:
a starch-derived polymer forming a hydrogel matrix;
a solvent comprising dimethyl sulfoxide, an aqueous solvent selected from phosphate buffered saline or sodium chloride solution, or a combination thereof; and
a therapeutic agent selected from a chemotherapeutic agent or a radionuclide, wherein the composition is a pre-formed injectable hydrogel formulation that is formed prior to administration;
optionally wherein the chemotherapeutic agent comprises doxorubicin in its free base form or a compound that is insoluble in water and soluble in dimethyl sulfoxide; and
optionally wherein the radionuclide is selected from an alpha-emitting radionuclide, a beta-emitting radionuclide, or a gamma-emitting radionuclide, and comprises cerium-144, cerium-141, actinium-225, thorium-227, lutetium-177, terbium-161, yttrium-90, or indium-111.

25. The composition of claim 24, wherein the therapeutic agent comprises both a chemotherapeutic agent and a radionuclide; optionally wherein the agents are co-retained in a single hydrogel matrix and administered simultaneously via a single intratumoral injection.

26. A method of treating a solid tumor in a subject comprising:
administering to the solid tumor a composition comprising:
a starch-based hydrogel comprising a network of starch polymer chains crosslinked using a crosslinking agent;
a solvent comprising dimethyl sulfoxide; and
a therapeutic agent selected from a chemotherapeutic agent or a radionuclide, wherein the composition is a pre-formed injectable hydrogel formulation that is formed prior to administration; optionally wherein the chemotherapeutic agent comprises doxorubicin in its free base form; optionally wherein the radionuclide is selected from cerium-144, cerium-141, actinium-225, thorium-227, lutetium-177, or terbium-161;
and optionally wherein the tumor comprises a pediatric brain tumor.
